(19) United States
(12) Patent Application Publication
(10) Pub. No.: US 2021/0290949 A1
(43) Pub. Date: Sep. 23, 2021

(54) NERVE CUFFS, METHODS OF FABRICATING THE SAME AND METHODS OF USE

(71) Applicants: GALVANI BIOELECTRONICS LIMITED, Brentford Middlesex (GB); TRUSTEES OF BOSTON UNIVERSITY, Boston, MA (US)

(72) Inventors: Bradley J. HOLINSKI, Brentford Middlesex (GB); Timothy GARDNER, Boston, MA (US); Charles A. LISSANDRELLO, Boston, MA (US); Alice E. WHITE, Boston, MA (US); Winthrop GILLIS, Boston, MA (US); Jun SHEN, Boston, MA (US); Timothy OTCHY, Boston, MA (US); Christos MICHAS, Boston, MA (US)

(73) Assignees: GALVANI BIOELECTRONICS LIMITED, Brentford Middlesex (GB); TRUSTEES OF BOSTON UNIVERSITY, Boston, MA (US)

(21) Appl. No.: 16/321,214

(22) PCT Filed: Jul. 27, 2017
(86) PCT No.: PCT/US2017/044096

§ 371 Date: Jan. 28, 2019

Related U.S. Application Data
(60) Provisional application No. 62/367,975, filed on Jul. 28, 2016.

(51) Int. Cl. A61N 1/05; B33Y 10/00; B33Y 80/00; B29C 64/124

(52) U.S. Cl. CPC A61N 1/0556; B33Y 10/00; B29L 2031/753; B29C 64/124; B33Y 80/00

(57) ABSTRACT

Implantable nerve cuffs and methods for constructing or manufacturing the same are provided. Also provided is a method for installing a nerve into a nerve passage in the nerve cuff and a system using the nerve cuff. The nerve cuff is configured to retain one or more signal carrying elements such as electrodes proximal to a peripheral nerve in a human or animal subject. The nerve cuff may be constructed using a 3D printing method.

### CROSS-REFERENCE TO RELATED APPLICATIONS

The present disclosure claims the benefit of U.S. Provisional Patent Application Ser. No. 62/367,975 filed Jul. 28, 2016, the entire content and disclosure of which is incorporated herein by reference.

### FIELD OF THE DISCLOSURE

The present disclosure relates to nerve cuffs for retaining one or more electrical signal carrying elements such as electrodes proximal to a peripheral nerve, and to methods of fabricating and using such nerve cuffs.

### BACKGROUND

Nerve cuffs for maintaining electrical interfaces with peripheral nerves in human and animal subjects have been developed for a variety of purposes. Such devices may be intended for implant into the body of a subject to provide a long term therapeutic effect through electrical stimulation or blocking of nerve activity, with frequently targeted nerves including the vagus nerve, the hypoglossal nerve, the sciatic nerve, and others. Such acute and chronic uses of electrical nerve stimulation include control of blood pressure, sleep disorders and diabetes, motor function control, and so forth. Such devices may also or instead be intended for use in recording electrical signals from a peripheral nerve, for example to assist in the delivery of a therapeutic effect, or for experimental purposes.

### SUMMARY

Disclosed are nerve cuffs configured to retain one or more signal carrying elements such as electrodes proximal to a peripheral nerve in a human or animal subject. Proximal used herein includes touching or having contact with the peripheral nerve.

In an aspect of the disclosure, the nerve cuff comprises a rigid cuff body having first and second ends, a nerve passage and an entry channel. The nerve passage extends between the first and second ends of the rigid cuff body. The nerve passage is configured to retain the peripheral nerve. The entry channel also extends between the first and second ends of the rigid cuff body. The entry channel guides the peripheral nerve towards the nerve passage. The nerve cuff is configured to inhibit the peripheral nerve from being removed or dislodged from the nerve cuff once the peripheral nerve is retained in the nerve passage.

In some aspects, the nerve cuff is further configured to bias the peripheral nerve towards the nerve passage.

In some aspects, the rigid cuff body comprises opposite first and second sides extending between the first and second ends. A portion of the first side and a portion of the second side form sidewalls of the entry channel. The sidewalls of the entry channel are angled such that an opening to the nerve passage from the entry channel is narrower in a transverse direction than an opening to the entry channel to an exterior in the transverse direction.

In some aspects, another portion of the first side and another portion of the second side form sidewalls of the nerve passage. The sidewalls of the nerve passage may be curved. The opening to the nerve passage from the entry channel is narrower in a transverse direction than a maximum distance between sidewalls in the transverse direction.

In some aspects, the entry channel is at least partly defined by entry channel side walls which approach the nerve passage such that the entry channel and the nerve passage define a neck between them. The neck is narrower than the nerve passage.

In some aspects, the nerve cuff has a small size and is capable of accepting or “cuff”-ing very small peripheral nerves for example with a diameter of less than about 2 mm, less than about 1 mm, less than about 500 μm, less than about 200 μm or less than about 100 μm. Thus, the nerve cuff may have a largest dimension of the cuff in a direction transverse to the nerve passage of less than about 2 mm, less than about 1 mm, less than about 500 μm, less than about 200 μm or less than about 100 μm. To accommodate such small peripheral nerves, the nerve passage may also be appropriately sized to retain the nerve without significant deformation, for example using a nerve passage having a diameter of less than about 2 mm, less than 1 about mm, less than about 500 μm, less than about 200 μm or less than about 100 μm.

In some aspects, the nerve cuff has a larger size and is capable of accepting or cuffing peripheral nerves for example with a diameter of about 5 mm or about 10 mm or larger.

In some aspects, the nerve cuff may be formed integrally of a single material, such as a polymer, for example a photopolymer used to construct the nerve cuff using a stereolithography (direct laser writing) process. The nerve cuff may additionally be coated, for example with a different polymer or other coating to provide modified surface characteristics such as biocompatibility or electrical conductivity on the interior or exterior of the cuff.

In some aspects, the rigid cuff body further comprises one or more electrode apertures extending through the rigid cuff body to the nerve passage for accepting one or more electrodes or other signal carrying elements, and to enable an electrode or other element to pass through the entire cuff body.

In some aspects, the one or more electrode apertures comprise at least one pair of electrode apertures. Each pair is arranged such that the electrode apertures of the pair respectively pass through a different side of the cuff body. The electrode apertures of the pair are aligned so that a single continuous electrode, for example, formed by a wire, or fibre can be retained in the two electrode apertures of a pair.

In some aspects, a plurality of pairs of electrode apertures may be distributed along the rigid cuff body between the first and second ends so that multiple electrodes can be provided to be proximal to distributed locations along a nerve when retained within the nerve passage.

In some aspects, the electrodes of the nerve cuff may comprise metallic wire, carbon nanotube bundles and fibers including nanowires, thin film electrodes and other materials, and may have a range of cross section dimensions, for example from about 1 to about 1000 μm, and various numbers of independent signal-carrying elements.

In some aspects, an electrode may be at least partly covered in an insulating layer where the part is external to the nerve cuff. The insulating layer is absent from at least a portion of the electrode within the nerve cuff.

In some aspects, the rigid cuff body of the nerve cuff may also be modified in such a way that the electrode or other signal carrying material substantially comprises or covers a substantial portion of the nerve passage. In such a configuration, one or more electrodes could be maintained proximal to the nerve by the opposing first and second elements and a retaining mechanism.

In some aspects, the nerve cuff further comprises a first segment and a second segment. The first segment and the second segment are connected to the rigid cuff body via connection pillars. The nerve cuff comprises a thin film electrode having a plurality of vias or gaps. The vias are dimensioned to allow the connection pillar to extend therethrough. The thin film electrode is disposed between the rigid cuff body and the first segment and the rigid cuff body and the second segment.

In some aspects, the thin film electrode comprises a plurality of electric contacts disposed in the nerve passage. The thin film electrode is proximal to the peripheral nerve when the peripheral nerve is in the nerve passage.

In some aspects, various additional features of the nerve cuff may be included in order to further help assist and/or bias the peripheral nerve along the entry channel towards the nerve passage, and/or to further help retain the nerve within the cuff or within the nerve passage. For example, one or more gating structures or trap-doors may also or instead be provided which are arranged to restrain or restrict movement of a nerve through the entry channel in a direction away from the nerve passage and/or to bias movement of a nerve through the entry channel in a direction towards the nerve passage. Such gating structures may be provided as part of the unitary structure of the nerve cuff, for example as components formed so as to be directly coupled to the cuff.

In some aspects, the gating structures may comprise one or more baffles protruding into the entry channel, and such baffles may be inclined towards the nerve passage so as to bias the movement of a nerve which is already at least partly within the entry channel towards the nerve passage.

In some aspects, the gating structures may comprise one or more flaps extending into the entry channel and inclined towards the nerve passage.

Such flaps may be coupled to the rigid cuff body so as to rotate around a resilient hinge portion of the flap which allows the flap to move under pressure from a nerve entering the cuff, but to subsequently return to a closed or unbiased position to resist exit of the nerve. Two such flaps may be provided in an opposed configuration so as to have proximal tips. The pair of flaps are arranged to separate when urged in a forward direction to permit a nerve to pass between the tips when moving through the entry channel towards the nerve passage.

In some aspects, when urged in a reverse direction the tips may be arranged to engage with each other to limit the reverse movement.

In some aspects, the tips may be arranged to interlock when engaged with each other.

In some aspects, the gating structures may comprise a lid. The lid may be arranged such that the lid can be moved between an open configuration to allow a nerve to pass into the entry channel towards the nerve passage, and a closed configuration in which the entry channel is blocked by the lid to prevent exit of a nerve out of the entry channel away from the nerve passage.

In some aspects, the lid may be hinged to provide movement between the open and closed configurations and the cuff may further comprise one or more catches formed on the cuff body to secure the lid in the closed configuration.

In some aspects, a combination of gating structures may be used.

In some aspects, the rigid cuff body may comprise a manipulator aperture. The manipulator aperture is configured to accept a manipulator tool, such as a needle, forceps or tweezer or the like, for handling the nerve cuff.

In some aspects, the manipulator aperture may extend in a direction transverse to the nerve passage, for example being located in a base of the cuff body distal from the opening of the entry passage. Further, in some aspects, the manipulator aperture may extend all the way across the rigid cuff body, opening on both sides, for example so that a manipulator tool can be accepted from either end of the aperture, or both ends at the same time to provide a more secure connection.

In some aspects, the manipulator aperture may intersect with the nerve passage. In some aspects, the nerve cuff may comprise a manipulator pad arranged for securing to a manipulator tool for example by gluing. The manipulator pad may be coupled to the rigid body by a frangible connection, so that the pad can be broken away from the nerve cuff with the manipulator tool.

In some aspects, the nerve cuff may have a flexible manipulator tab that may be either secured to a manipulator tool by gluing or simply mechanically gripped. The tab may be permanently affixed to the cuff and released from the manipulator after implant, or may be broken away from the cuff after implant. In some aspects a combination of pad, tab and aperture may be used.

Disclosed are also methods for constructing or manufacturing a nerve cuff as set out above. In some aspects, a method of constructing or manufacturing a nerve cuff being configured for retaining one or more electrodes or other signal carrying elements proximal to a peripheral nerve when retained within the cuff, the method comprising forming the nerve cuff as an integral unit comprising at least the rigid cuff body having the opposite first and second ends, the nerve cuff being configured to inhibit the peripheral nerve, when retained in the nerve passage, from being removed from the nerve cuff.

In some aspects, some or all of the nerve cuff may be constructed using a 3D printing technique, for example stereolithography or direct laser writing technique in which a light field is used to write the cuff structure into a photopolymer. The nerve cuff may be further treated after printing, for example by coating with a material to modify particular surface properties such as biocompatibility or conductivity, for example using iridium oxide, PEDOT:PSS (poly(3,4-ethylenedioxythiophene) polystyrene sulfonate), parylene or a similar polymer.

In some aspects, the method of constructing or manufacturing may comprise forming a portion of the cuff comprising one or more open channels each corresponding to an uncompleted electrode aperture; laying one or more electrodes into the open channels; and forming a further portion of the nerve cuff on the portion thereby closing the one or more open channels to complete the electrode apertures containing said one or more electrodes.

In some aspects, the method further comprises forming a manipulator aperture as an opening in the portion of the nerve cuff.

In some aspects, the method further comprises forming a roughened surface on the portion of the nerve cuff. The roughened surface acts as an interface for forming the further portion of the nerve cuff.

In some aspects, the method further comprises aligning in a direction transverse to the nerve passage, a pair of open channels. The pair of open channels corresponds to the apertures. The method further comprises aligning in the direction transverse to the nerve passage an electrode.

In some aspects, a method for constructing or manufacturing a nerve cuff may comprise placing a thin film electrode at a set distance from a substrate, forming a first segment and a second segment between the thin film electrode and the substrate, forming connection pillars through a one or more vias in the thin film electrode, on the first segment and the second segment; and forming a rigid cuff body having opposite first and second ends and sidewalls extending between the first end and the second end. At least a portion of the sidewalls is configured and dimensioned to provide the nerve passage.

The first segment and the second segment have at least a portion separate from each other in a direction transverse to the nerve passage.

In some aspects, the rigid cuff body may be formed around the electrodes in a single step by a stereolithography or direct laser writing process that is capable of polymerizing photopolymers beneath, through, around and above the electrode in a single fabrication step.

In some aspects, the one or more signal carrying elements such as electrodes, are positioned within the nerve cuff such that when the peripheral nerve is within the nerve passage the one or more signal carrying elements can be used to deliver and/or receive one or more signals to and/or from the peripheral nerve.

The cuff body may be referred to as a cuff body block, and may be described as a rigid cuff body, being constructed such that the opposing first and second sides substantially retain their shape and configuration during normal use, including when a nerve is being introduced into the cuff body.

In some aspects, a method of retaining one or more electrodes proximal to a peripheral nerve comprises providing a nerve cuff as set forth above and moving the peripheral nerve through the entry channel and into the nerve passage so as to be retained proximal to the one or more electrodes.

In some aspects, moving and positioning may be carried out using a manipulator coupled to the rigid cuff body, and may then further comprise removing the manipulator when the nerve is retained proximal to the one or more electrodes or other signal carrying elements.

In some aspects, the moving may be facilitated by a flexible tab coupled to the rigid body, and may or may not then be detached when the nerve is so retained.

In some aspects, the method further comprises at least one of reading an electrical or other signal from the nerve, and passing an electrical or other signal to the nerve, using the one or more electrodes or other signal carrying elements.

The term “a nerve” or “a peripheral nerve” used herein, may also refer to a branch of a peripheral nerve, or a ganglion containing the cell bodies of the nerve and may also refer to a part of a nerve (including part of a branch) which has been separated out, for example a dissected fascicle or other subcomponent of a nerve, in a manner which permits only that part of the nerve to be introduced into the nerve cuff.

Where particular directions might be implied by language used to describe the nerve cuffs and their use, such as top, base, sides, up, down, laterally, and similar, these should be understood as for convenience of description only, since a nerve cuff may be constructed, manipulated and installed in any preferred or convenient orientation.

### BRIEF DESCRIPTION OF THE DRAWINGS

FIG. 1 illustrates a perspective view of a nerve cuff which comprises a nerve passage and an entry passage according to aspects of the disclosure;

FIGS. 2 to 4 illustrate nerve cuffs which comprise a pair of resiliently hinged flaps providing gating structures according to aspects of the disclosure;

FIGS. 5 and 6 illustrate nerve cuffs comprising baffle structures according to aspects of the disclosure;

FIGS. 7 and 8 illustrate nerve cuffs comprising lid structures according to aspects of the disclosure;

FIG. 9 illustrates a nerve cuff which comprises a manipulator tool pad according to aspects of the disclosure;

FIGS. 10a-10d illustrate a method constructing a nerve cuff in accordance with aspects of the disclosure;

FIG. 11 is an electron micrograph of a completed nerve cuff with electrodes installed using the method illustrated in FIG. 10;

FIGS. 12a-12d illustrates a method of installing a peripheral nerve within a nerve cuff in accordance with aspects of the disclosure;

FIG. 13 illustrates a system comprising a nerve cuff in accordance with aspects of the disclosure installed in a human or animal subject;

FIGS. 14a-14d illustrate different views of a nerve cuff in accordance with aspects of the disclosure that can accommodate a thin film electrode as illustrated in FIG. 14d; and

FIGS. 15a-15e illustrate a method of constructing the nerve cuff of FIG. 14 in accordance with aspects of the disclosure.

### DETAILED DESCRIPTION

As used herein, the recitation of a numerical range for a variable is intended to convey that the variable may be equal to any of the values within that range. Thus, for a variable which is inherently discrete, the variable may be equal to any integer value in the numerical range, including the end-points of the range. Similarly, for a variable which is inherently continuous, the variable may be equal to any real or imaginary value of the numerical range, including the endpoints of the range. As an example, a variable which is described as having values between 0 and 2, may include 0, 1 or 2 for variables which are inherently discrete, and may include 0.0, 0.1, 0.01, 0.001, or any other real or imaginary value for variables which are inherently continuous.

In the present disclosure, the term “preferably” or “preferred” is non-exclusive where it is intended to mean “preferably, but not limited to”. Any steps recited in any method or process claims may be executed in any order and are not limited to the order presented in the claims. Means-plus function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the disclosure should be determined solely by the appended claims and their legal equivalents, rather than by the descriptions and examples given herein.

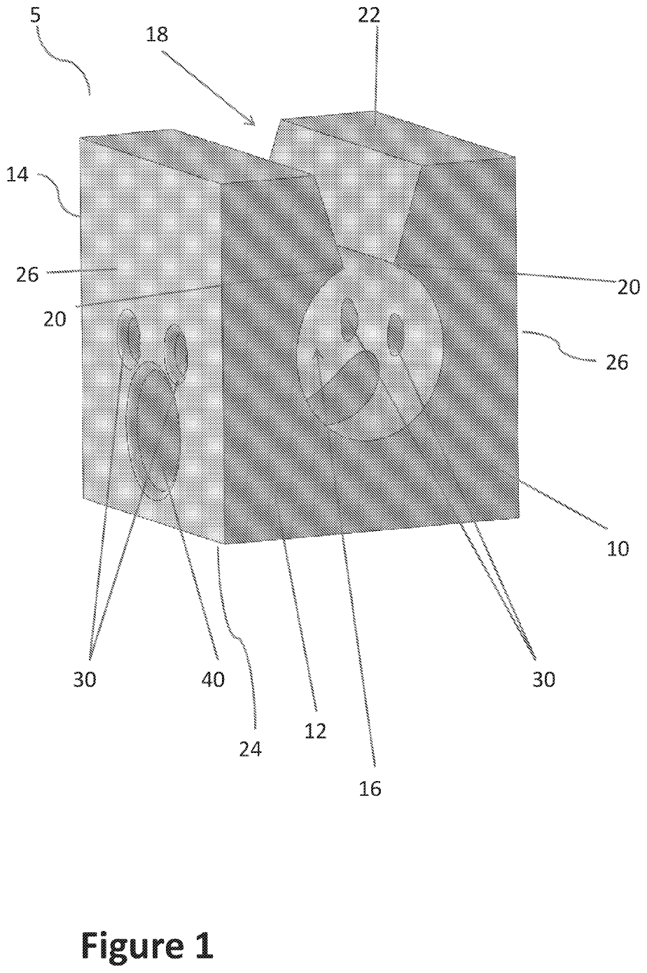

FIG. 1 illustrates a nerve cuff 5 for retaining one or more electrodes. A nerve cuff, such as nerve cuff 5, is typically used to provide electrical stimulation to a nerve and/or to detect electrical signals from the nerve. The nerve cuff 5 comprises a cuff body 10 which is constructed so as to substantially retain its shape and configuration during normal use, including when a nerve is being introduced into the cuff body, and which may therefore be referred to as rigid, so that the cuff body is a rigid cuff body 10. Of course, some minor degree of deformation of the cuff body may take place during such use, and as discussed below, various more flexible elements may be appended to the cuff body such as baffles, hinged elements, and flexible signal carrying elements for other purposes.

The cuff body 10 comprises first and second opposite ends 12, 14, and a nerve passage 16 extends along a nerve axis through the cuff body 10 between these opposite ends for retaining a nerve (not shown in this figure) along this nerve axis (when the nerve is installed) (hereinafter a direction in which the nerve passage 16 extends). A direction transverse to the direction in which the nerve passage 16 extends is perpendicular to this direction).

An entry channel 18 is provided in the cuff body 10 such that an in vivo nerve can be introduced laterally through the entry channel 18 and into the nerve passage 16 preferably without damaging the nerve. In the orientation of FIG. 1 this is laterally downwards although of course the orientation in practice will depend in the orientation of the in vivo nerve. Some deformation of the nerve when passing through the entry channel may be expected, because the cuff may be proportioned such that the nerve needs to be “squeezed” through the entry channel 18 to arrive in the nerve passage 16. The nerve passage 16 may be suitably proportioned and shaped to retain the nerve in a substantially or minimally deformed state, to minimize any adverse physiological effects on the nerve, and to this end a nerve cuff of suitable size may be selected or specially fabricated for use on a particular nerve.

The nerve cuff 5 illustrated in FIG. 1 and described elsewhere in this document may be generally rectilinear or cuboid in form as shown in FIG. 1, but other forms, such as other prismatic or geometric shapes (circular, elliptic, etc.) extending between the first and second ends, may be used. As shown in FIG. 1, the first and second ends 12, 14 of the cuff body 10 may be provided by first and second end faces of the cuff body 10, with the nerve passage 16 and entry channel 18 extending between and typically transversely or substantially orthogonally to these end faces.

The entry channel 18 may be in the form of a linear slot in the cuff body for accepting the in vivo nerve laterally into the nerve passage 16, and to this end the entry channel may typically be substantially linear and parallel to the nerve passage and nerve axis. The entry channel 18 may typically be formed in a top 22 of the cuff body opposite to a base 24 of the cuff body, as illustrated in FIG. 1, wherein the top 22 and base 24 are joined by cuff body sides 26 (first side and second side). The nerve passage 16 may typically be of circular or elliptic cylindrical form so as to match a typical cross sectional shape of a nerve to be brought into the cuff.

In accordance with aspects of the disclosure, the nerve cuff 5, including the rigid body, but not necessarily including any electrodes to be used with the cuff, may be formed as a single, integral unit, and optionally from a single material such as a polymer, for example using construction techniques described in more detail below.

In accordance with aspects of the disclosure, various different arrangements may be used to help bias the nerve through the entry channel 18 and into the nerve passage 16, and/or to help retain the nerve within the nerve passage 16 or more generally within the nerve cuff 5, and some of these arrangements are discussed later in this specification. In the example of FIG. 1, these aims are addressed by shaping the cuff body 10 such that the opening of the entry channel 18 into the nerve passage 16 is narrower than the nerve passage as a whole, providing a neck 20, so that a nerve which is of larger diameter than this neck 20 when the nerve is in an undeformed state will tend to be retained in the nerve passage 16. In aspects of the disclosure, as illustrated in FIG. 1, portions of the cuff body sides 26 form the sidewalls of the entry channel 18 and nerve passage 16. The portion of the cuff body sides 26 that form the sidewalls of the entry channel 18 are angled. Thus, the cuff body sides 26 do not have the same thickness from the top 22 to the base 24. For example, as illustrated in FIG. 1, as the distance from the top 22 increases, the portions of the cuff body sides 26 forming the sidewalls of the entry channel 28 become thicker.

In FIG. 1, the portions of the cuff body sides 26 forming the sidewalls of the entry channel 18 also converge towards the nerve passage 16 to better define the neck 20, and to aid in gradually deforming the nerve as it is obliged through the entry passage 18 before emerging into the nerve passage 16.

In aspects of the disclosure, the nerve cuff 5 is configured to retain one or more electrodes proximal to the nerve. This can be achieved in various ways. For example, as illustrated in FIG. 1 the cuff body 10 may comprise one or more electrode apertures 30. These electrode apertures 30 extend from an outside of the cuff body 10 at least through to the nerve passage 16. FIG. 1 illustrates two such apertures in each side 26 of the cuff body. Of these, an electrode aperture in one side 26 is aligned as a pair with a corresponding aperture in the other side 26 of the cuff body such that a substantially straight electrode can pass through one side, across the nerve passage 16, and through the other side of the cuff body. In this way, the nerve cuff 5 illustrated in FIG. 1 can be used to retain two electrodes proximal to a nerve when retained within the nerve passage, each electrode passing through both of the electrode apertures of each pair.

Of course, different numbers of electrode apertures can be provided into the nerve passage 16, for example three, four, or more such electrodes. Typically, such electrodes may be aligned in a substantially straight line perpendicular to the nerve along the cuff body, but this need not be the case. In another aspect of the disclosure, the electrode apertures can be provided singly, rather than in pairs, such that an electrode can pass through such an aperture to the nerve passage 16, but does not continue into an opposite or aligned electrode aperture at the other side of the nerve passage 16. Although the electrode apertures 30 of FIG. 1, and therefore the corresponding electrodes, are illustrated as aligned substantially orthogonally or transversely to the nerve passage 16 and axis of the nerve to be retained, and are also illustrated as being aligned in the plane of the base 24, other angles and orientations can be used, and electrode apertures may also or instead be provided in the top 22, base 24, end faces and any other suitable part of the cuff body 10.

In another aspect of the disclosure, the cuff body 10 further comprises a manipulator aperture 40. The manipulator aperture 40 is arranged for receiving a needle or other manipulator tool (not shown in FIG. 1) for handling the cuff 5, for example for use in bringing the cuff 5 into position near a peripheral nerve, and for obliging the nerve into the nerve passage through the entry channel. When the nerve is in place, the manipulator tool is then typically withdrawn from the cuff.

Although the manipulator aperture 40 of FIG. 1 passes entirely through a lower region of the cuff body (i.e. a region distal from the top 22 in which the entry channel is formed), from one side to the other side of the cuff body transversely to the nerve passage 16, in other aspects of the disclosure, the manipulator aperture 40 could be an aperture not emerging from both sides of the body, and could be oriented and positioned in various other ways. In order to provide a manipulator aperture 40 of sufficient diameter for a suitably strong manipulator tool if the nerve cuff 5 is very small, the manipulator aperture 40 of FIG. 1 may at least partly intersect with the lower part of the nerve passage, but could instead be located entirely in the base 24 of the cuff body without intersecting with the nerve passage, or could be formed in some other part of the cuff body 10.

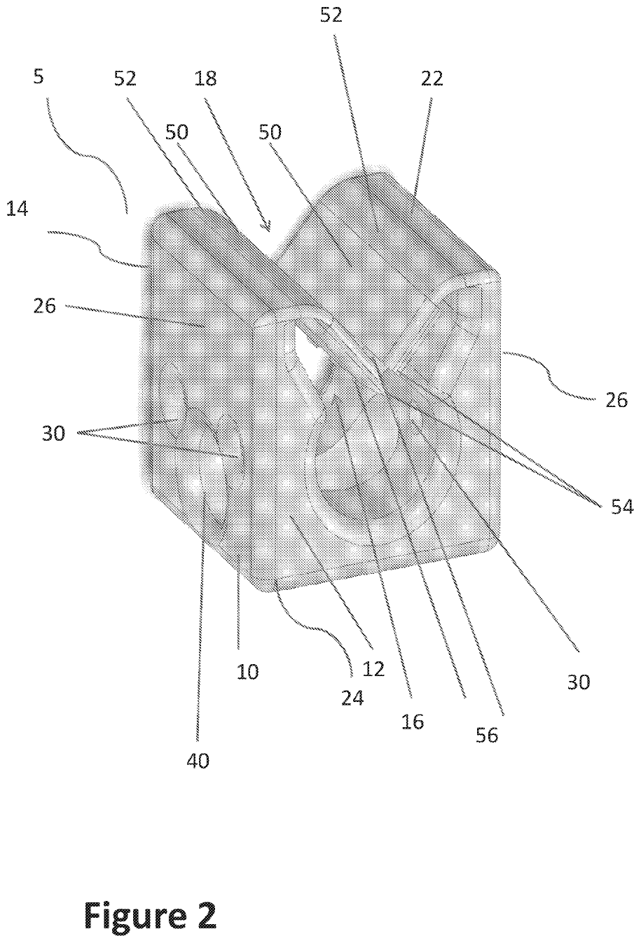

In the arrangement of FIG. 1, a neck 20 is defined between the entry channel 18 and the nerve passage 16 in order to help retain a peripheral nerve within the nerve passage 16. According to other aspects of the disclosure, various other arrangements may be used to help bias the nerve through the entry channel 18 and into the nerve passage 16, and/or to help retain the nerve within the nerve passage 16 or more generally within the nerve cuff 5, and some particular such arrangements are illustrated in FIGS. 2-9, along with some other options for design and configuration of the nerve cuff 5.

In particular, the nerve cuff 5 may additionally comprise one or more gating structures for achieving these aims. FIG. 2 shows a nerve cuff 5 which is similar to that illustrated in FIG. 1, in which such gating structures are provided by a pair of opposing hinged flaps 50 in accordance with aspects of the disclosure. Each flap 50 extends from the top 22 of the cuff body 10 into and partly across the entry channel 18, and is coupled to the cuff body 10 at the top 22 of the body by a resilient hinge portion 52 of the flap which permits each flap to rotate about the hinge portion 52 under an applied force. When a peripheral nerve is obliged into the entry channel 18 against the flaps 50, the flaps 50 are urged by the peripheral nerve (not shown in FIG. 2), against the resilience of the hinge portions 52, further into and therefore also nearer to the sides 26, until the peripheral nerve is able to pass between the flaps 50 and into the nerve passage 16.

Each flap 50 also has a tip portion 54 distal from the respective hinge portion 52. When the peripheral nerve has passed through the gating structures provided by the flaps 50, the resilience of the hinge portions 52 and/or any backward movement by or pressure from the peripheral nerve now in the nerve passage 16 tends to urge the flaps 50 in a reverse direction. However, a combination of one or more of the inclination of the flaps in the entry channel 18 downwards towards the nerve channel, the resilience of the hinge portions 52, and confrontation between the tip portions 54 of the flaps, at least resist, and preferably also block or limit rotation of the flaps 50 in the reverse direction, thereby retaining the peripheral nerve within the cuff 5.

The flaps 50 effectively acts as sidewalls of the entry channel 18.

The tip portions 54 of the flaps 50 may therefore be said to be arranged to come into confrontation to lock together when urged in a reverse direction away from the nerve passage 16, blocking further rotation of the flaps 50. This may be achieved by a simple confrontation of the tip portions, but additionally the flaps may be constructed and arranged such that the tip portions 54 are provided with interlocking structures, such as the interlocking teeth 56 of FIG. 2, to provide a more secure locking action.

The hinge regions 52 of the flaps 50 may be provided in various ways. In the arrangement of FIG. 2, these are shown as formed from thinner areas of material of the cuff 5 which therefore provide increased flexibility for bending, with the main portions of the flaps beyond the hinges being provided by thicker areas of material of the cuff 5. Another way of improving the flexibility of the hinge portions 5 may be to provide apertures though the material of the flaps in the hinge portions; however, various other constructions could be used.

The cuff 5 of FIG. 2 also comprises two pairs of electrode apertures 30 and a single manipulator aperture 40 in the same configuration and for the same purposes as those shown in FIG. 1. Therefore, the apertures 30 and 40 will not be described again with respect to FIG. 2.

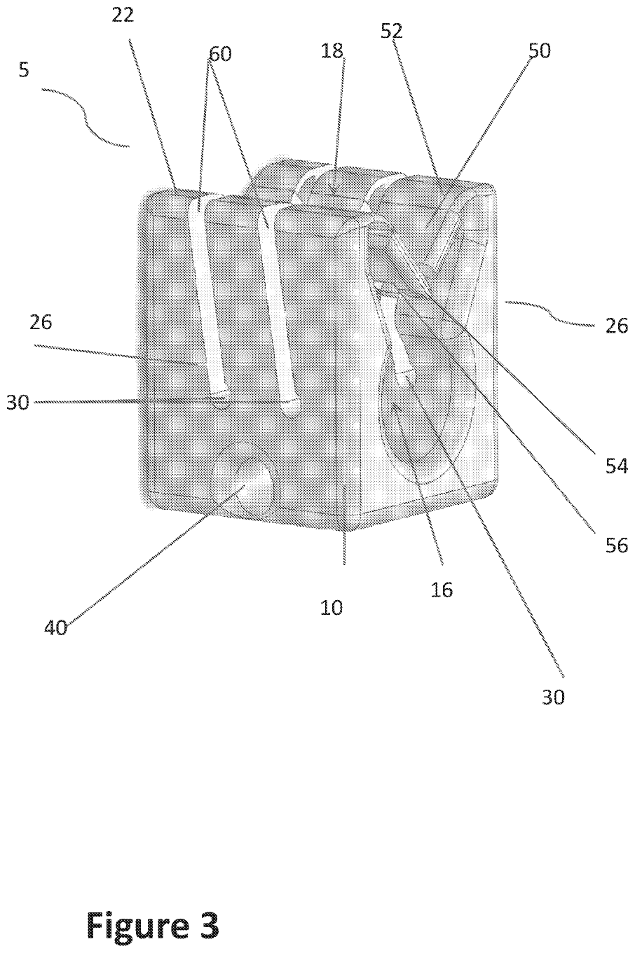

FIG. 3 illustrates a nerve cuff 5 in accordance with aspects of the disclosure. Many of the features in the nerve cuff 5 of FIG. 3 have been described with respect to FIGS. 1 and 2 and therefore, will not be described again. The cuff body 10 of the nerve cuff 5 illustrated in FIG. 3 comprises electrode slots 60, to facilitate insertion of electrodes into the electrode apertures 30. The electrode apertures 30 are formed at the end of electrode slots 60. These electrode slots 60 extend from the top 22 of the cuff body 10, down through both the cuff body 10 and any gating structures such as the flaps 50, to the electrode apertures 30. These electrode slots 60 then permit electrodes to be passed through the cuff 5 and into the electrode apertures 30, for example, after fabrication of the cuff 5 is completed. If the electrode apertures 30 are provided in aligned pairs extending between the sides of the cuff body 10, then the slots 60 may also be provided in corresponding aligned pairs, so that a single electrode can be dropped into two aligned slots to be located in two aligned electrode apertures 30.

The dimensions of the electrode slots 60 can be tailored to a specific electrode being inserted. For example, a wider electrode would have a wider slot.

In another aspect of the disclosure, the electrode slots 60 can be included in the nerve cuff illustrated in FIG. 1. The electrode slots 60 would be formed in the cuff sides 26 and top 22.

Other ways of introducing electrodes into the cuff 5 during construction of the cuff, without requiring such electrode slots 60 to be provided, are discussed later in this document.

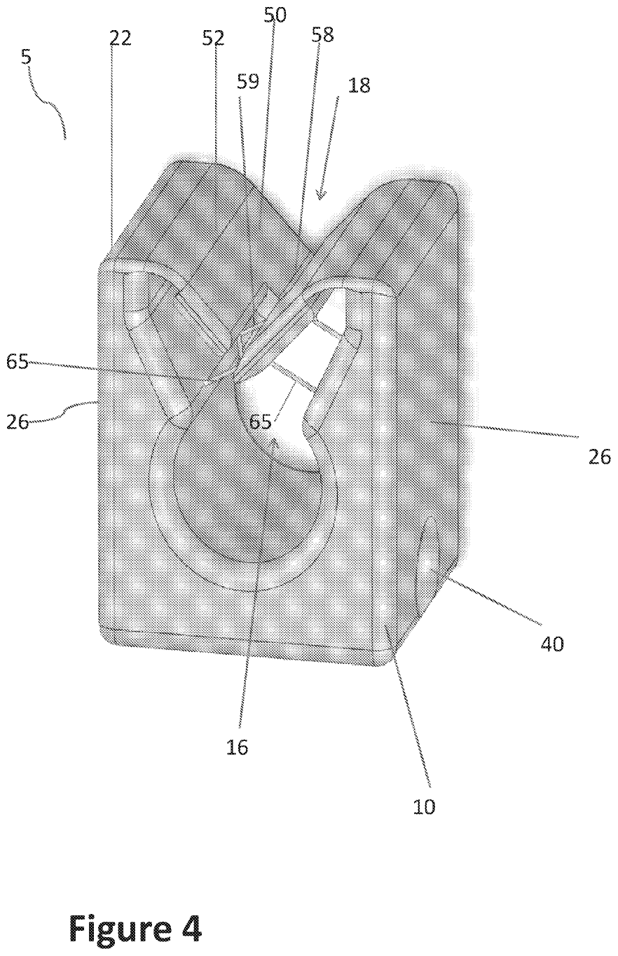

FIG. 4 illustrates a nerve cuff 5 in accordance with aspects of the disclosure. Many of the features in the nerve cuff 5 of FIG. 4 have been described with respect to FIGS. 1-3 and therefore, will not be described again. In this Figure, electrode apertures are omitted for clarity, but may be provided if required in various ways including as discussed above. A single manipulator aperture 40 is shown which is similar to that illustrated in FIG. 2, but which is provided entirely within the material of a base portion of the cuff body 10 without intersecting with the nerve passage 16. In FIG. 4, gating structures are provided as a pair of hinged flaps 50 in a similar manner to those shown in FIGS. 2 and 3, but instead of the tip portions 54 of the flaps 50 being provided with interlocking teeth 56, an extended half 58 of each tip portion provides an interlock with a corresponding truncated half 59 of the opposing tip portion. Of course, each tip portion 50 could have more than one extended region and more than one truncated tip region, as long as the extended and truncated regions of the two flaps 50 provide the required interlock.

In another aspect of the disclosure, the cuff 5 comprises one or more support rods 65 as illustrated in FIG. 4. It is noted that the support rods 65 may be added to and used with other constructions of the cuff 5 discussed herein. The support rods 65 can have different thickness, but may be a fine support rod. The support rods 65 extend or protrude from the sides 26 of the nerve cuff body 10. The support rods 65 may be flexible to allow for movement of the flaps 50. Additionally, for example as part of the stereolithographic processes discussed below, in order to provide support for the flaps during the construction process, and may be omitted, included or modified for example depending on the construction process, the orientation of the cuff 5 during construction, and other factors.

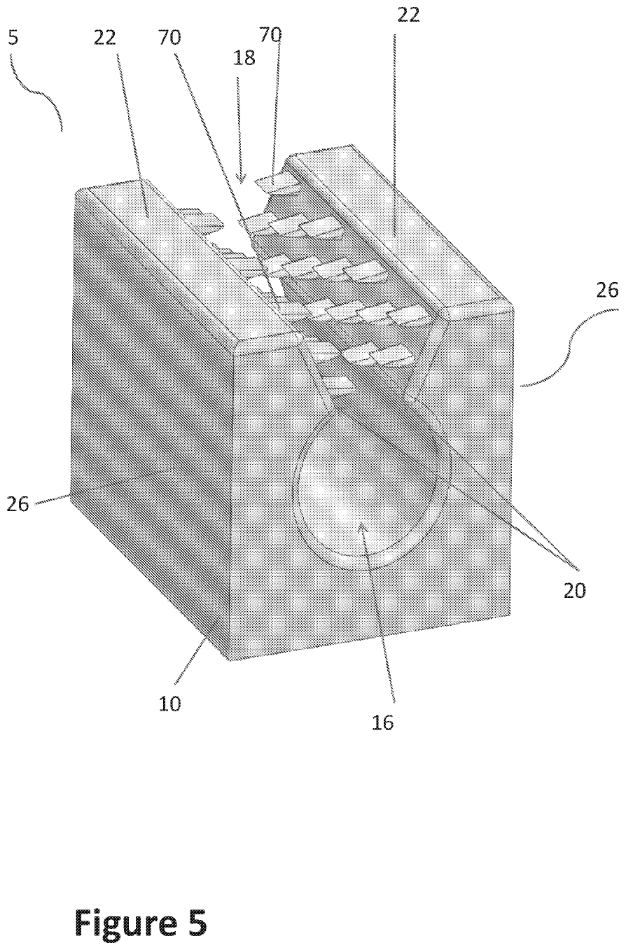

FIG. 5 illustrates a nerve cuff 5 in accordance with aspects of the disclosure. The nerve cuff 5 illustrated in FIG. 5 comprising one or more baffles protruding in the entry channel 18 as its gating structures instead hinged flaps 50. These baffles extend from the side walls of the entry channel 18 (e.g., portions of the sides 26 of the cuff body 10), and serve to restrain movement of a nerve through the entry channel 18 in a direction away from the nerve passage 16 and/or to bias movement of a nerve through the entry channel 18 in a direction towards the nerve passage 16. For example, the one or more baffles may be inclined in a direction towards the nerve passage 16, so as to improve bias of movement towards the nerve passage 16.

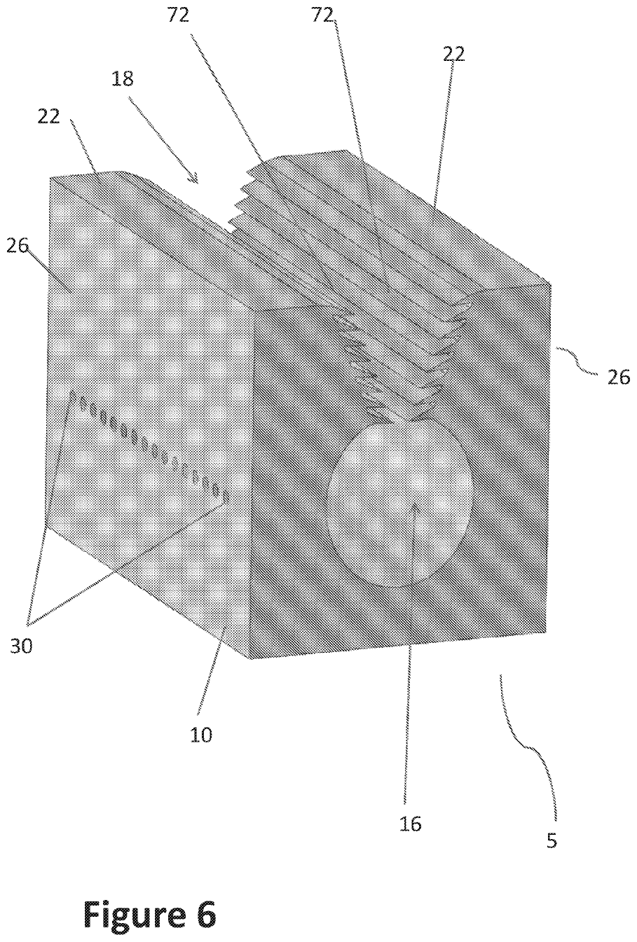

In the arrangement of FIG. 5 the baffles are provided by about fourteen teeth 70 extending from each side wall 26 of the entry channel 18, and these teeth 70 are distributed in an array of four rows but are short enough to leave a central gap through which a peripheral nerve may be obliged to pass. However, various different numbers, shapes, and distributions of such baffles may be used. For example, in FIG. 6 the baffles are provided by a series of parallel elongate ridges 72 each extending along the full length of the entry channel 18 between the end faces 12, 14 of the cuff body 10. In the illustrated example in FIG. 6, each ridge is inclined slightly towards the nerve passage 16 to assist in biasing movement of a peripheral nerve within the entry channel 18 towards the nerve passage 16.

In FIGS. 5 and 6 no manipulator aperture is shown, but could be provided if required. FIG. 5 also omits any electrode apertures, but these could be provided as desired in accordance with any of the aspects of the disclosure. The electrode apertures 30 shown in FIG. 6 pass only through one side of the cuff body 10 to the nerve passage 16, and there are no corresponding paired and aligned apertures in the other side of the cuff body 10. FIG. 6 also shows how a larger number of electrode apertures may be used, in this case about sixteen electrode apertures 30 distributed in a straight line such that the electrodes may be perpendicular to the nerve passage, along a side 26 of the cuff body 10.

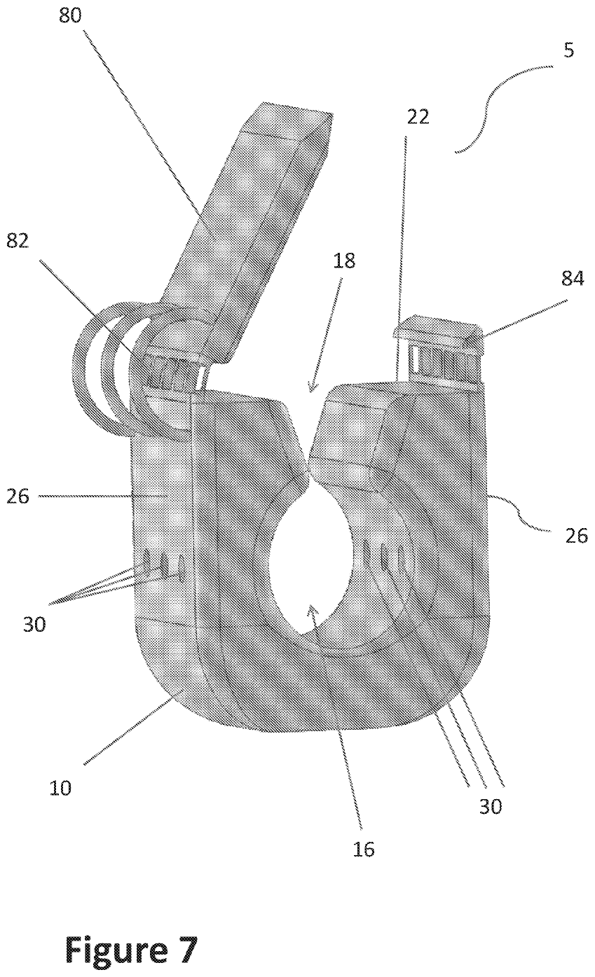

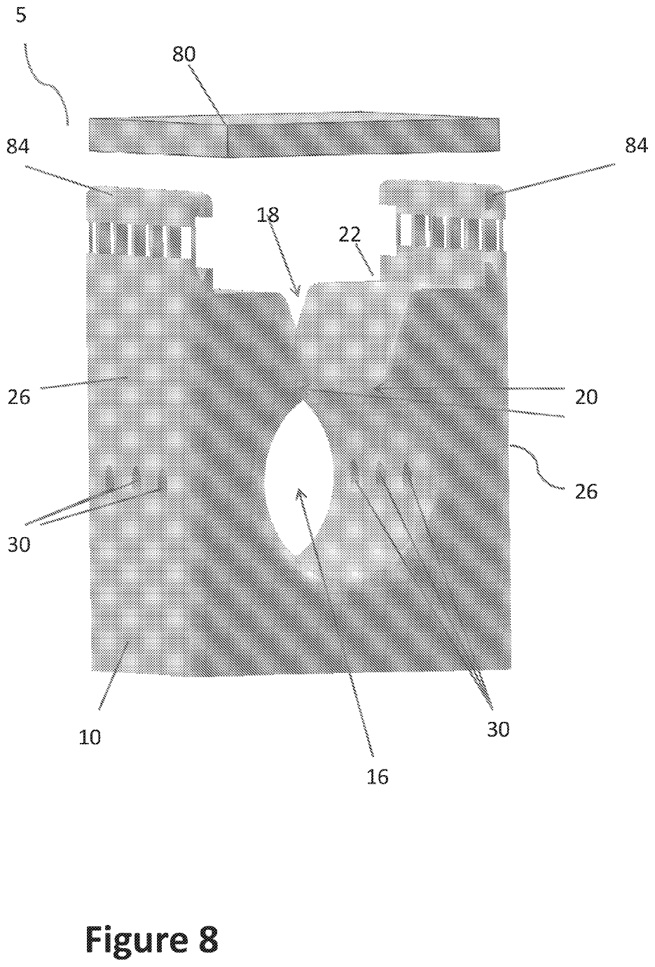

FIGS. 7 and 8 illustrate nerve cuffs 5 in accordance with aspects of the disclosure. Many of the features in the nerve cuffs 5 of FIGS. 7 and 8 have been described above and therefore, will not be described again. The manipulator aperture has also been omitted from FIGS. 7 and 8, but could be provided if required in accordance with the any of the aspects of the disclosure. FIGS. 7 and 8 show additional or alternative gating structures which may be used in combination with or separately to other gating structures described herein. In both FIGS. 7 and 8 the gating structure is a lid 80 which is arranged such that the lid can be moved between an open configuration to allow a nerve to pass into the entry channel 18 towards the nerve passage 16, and a closed configuration in which the entry channel 18 is blocked by the lid 80 to prevent exit of a nerve out of the entry channel 18 away from the nerve passage 16. In both FIGS. 7 and 8, the closed configuration is provided where the lid is located on the top 22 of the cuff body 10, for example covering substantially the hole of the top. In FIG. 7, the lid 80 is coupled to the cuff body 10 at one side of the top of the body by a lid hinge 82, and a lid catch 84 is disposed at an opposite side of the top of the body such that when the lid 80 is pressed into a closed configuration the lid catch 84 retains the lid 80 in this position. This clipping action may be designed to be irreversible, for example by means of the lid catch 84 being provided with a top surface oblique to the lid closure movement to allow a tip of the lid to push past into a closed configuration, and a lower surface perpendicular to the reverse lid movement to prevent the tip from pushing back towards the open configuration.

In an aspect of the disclosure, the lid 80 may be positioned with a tool. For example, the lid 80 may be manipulated with forceps, tweezers or other standard surgical instrument to press the lid 80 into the closed configuration. In another aspect of the disclosure, the lid 80 comprises a frangible, break-away connection, coupled to a manipulator (the frangible, break-away connection and the manipulator is not shown in the figure).

The frangible, break-away connection is designed to be strong enough for the lid 80 to be handled and closed onto the top, e.g., pressed into the closed configuration and engaged with the lid catch 84, but weak enough that a subsequent movement or action such as a twisting action causes the frangible connection to break, allowing the manipulator to be separated from the lid 80.

In FIG. 8, the lid is not hinged, but is instead provided as a separate component which may be slid into place from the side, or pressed into place from above so as to be retained by lid catches 84 at either side of the top 22. Similar to above, the lid may be moved or manipulated with forceps or other standard surgical instrument to close the latch, e.g., pressing the lid into place so as to be retained by the lid catches 84.

As described above, the gating structures may be combined. For example, baffles can be employed with flaps 50. The baffles would extend from the flaps 50 instead of sides 26.

In other examples, baffles can be combined with a lid 80, or flaps 50 combined with a lid 80.

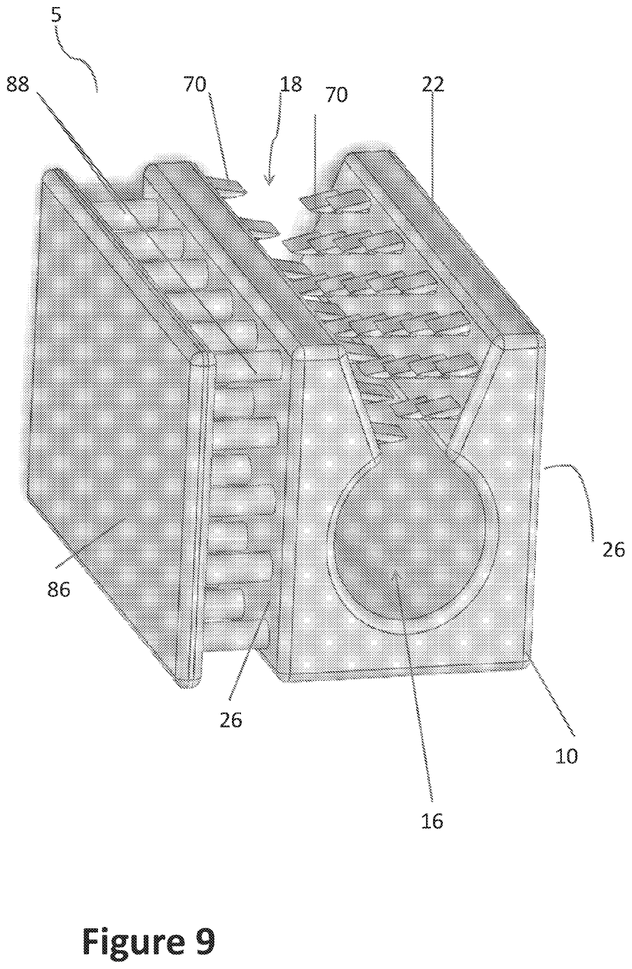

FIG. 9 illustrates a nerve cuff 5 in accordance with aspects of the disclosure. For clarity, the electrode aperture 30 has been omitted from FIG. 9, however, the electrode aperture 30 can be provided in accordance with aspects of the disclosure. Many of the features in the nerve cuff 5 illustrated in FIG. 9 have been described above and therefore, will not be described again (e.g., nerve passage 16, entry channel 18, cuff body 10, and the teeth as the baffles 70).

FIG. 9 shows an alternative or additional structure for accepting a manipulator tool, in which a manipulator pad 86 is coupled to the cuff body 10 by a frangible connection 88. While FIG. 9 shows teeth as the baffles for the gating structure, any other gating structure in accordance with aspects of the disclosure can be used instead or in combination. Additionally, although FIG. 9 shows a gating structure, a gating structure can be omitted from the nerve cuff 5 depicted in FIG. 9.

The frangible connection 88 is designed to be strong enough for the cuff 5 to be handled and applied to a peripheral nerve, but weak enough that a subsequent movement or action such as a twisting action causes the frangible connection 88 to break, allowing the manipulator pad 86 to be separated from the cuff body 10. This arrangement can then be used, in practice, by bonding, for example with a glue, or otherwise coupling, a manipulator tool such as a fine rod or needle with a suitably shaped end, to the manipulator pad. When the manipulator tool is no longer needed following installation of the cuff 5, a user then shears the manipulator tool, still coupled to the pad, away from the cuff.

The frangible connection 88 can be provided in various ways, but in FIG. 9 it is provided by an array of short columns connecting the manipulator pad 86 to the cuff body 10. The shape of the columns may be changed. Although in FIG. 9 the manipulator pad 86 is shown coupled to a side of the cuff body, in other aspects of the disclosure, it may instead be desirable to couple the pad 86 to the base of the cuff body in order to provide space for electrode apertures 30, and/or or to provide a smaller manipulator pad 86.

In an aspect of the disclosure, the manipulator pad 86 may be attached to the cuff body 10 via the frangible connection 88 after the electrodes are inserted into the electrode apertures 30.

The various cuffs 5 as described herein may be created using a variety of different processes and techniques. An example of such a process or technique is to form the cuff using any of a variety of 3D printing techniques. Various suitable 3D printing techniques are known, but one such suitable technique is to use stereolithography, in which a light field (typically provided by one or more laser beams) is used to write the required cuff structure into a photopolymer liquid which hardens in selective locations (or voxels) under influence of the light field.

Certain reference numbers have been omitted from FIGS. 2-9 for clarity of the figures. However, even though the reference numbers have been omitted, the cuffs may have the features.

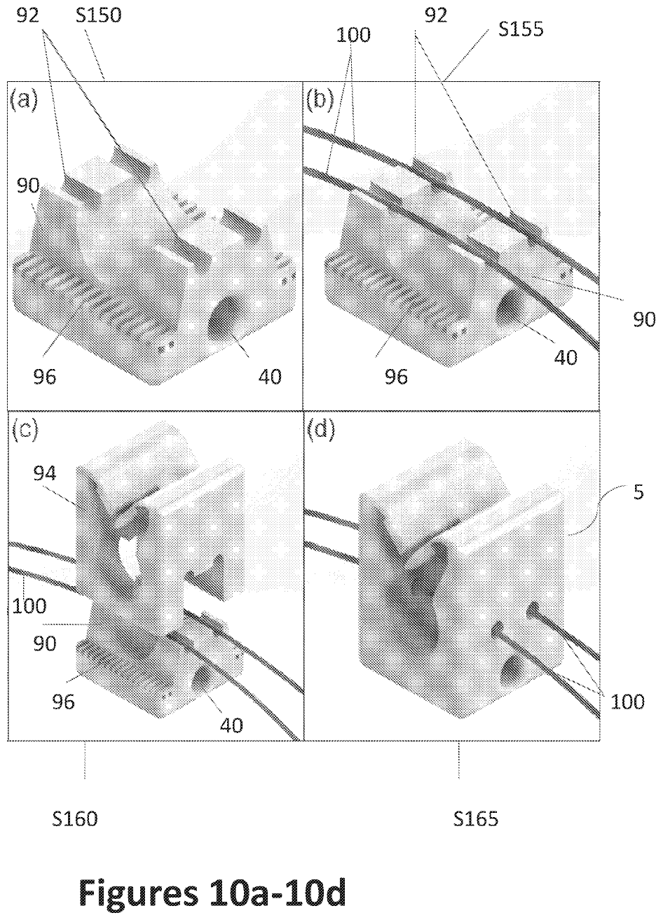

FIGS. 10a-10d illustrate a method for constructing or manufacturing a nerve cuff having one or more electrodes in accordance with aspects of the disclosure. As shown in FIG. 10a, a portion 90 of the nerve cuff 5 is first constructed at S150, up to and including a part of at least one, and typically part of each of the electrode apertures 30. In an aspect of the disclosure the nerve cuff 5 is formed from a photopolymer material, e.g., liquid, using a lithography system. The lithography system scans a focused laser beam into a droplet of commercially available, UV-curable polymer. Initially, the curable polymer is deposited on a substrate. The substrate may be a silicon or glass substrate. However, other substrates may be used.

In an aspect of the disclosure, the portion is formed layer-by-layer using the focused laser beam to cure a voxel of the polymer. As illustrated in FIG. 10a, the open channels 92 were provided by two pairs of aligned semi-cylindrical cut-outs each with a diameter of around 35 μm and a separation of around 150 μm, to provide alignment for the placement of two conducting electrodes 100. The cut-outs are formed by controlling the laser not to illuminate the area where the cut-outs are intended. Thus, the UV-curable polymer will remain in a liquid form and be subsequently removed, e.g., washed away. The diameter of the cut-outs and separation is only provided, by way of example, and other dimensions and separation may be used.

In an aspect of the disclosure, some surfaces or steps of the portion 90 were provided with a roughened surface 96 such as a cross-hatch design to increase the surface area for improved bonding with the portion 94 when subsequently fabrication on the same. The cross-hatch design is created in a similar manner as described above, e.g., controlling the laser not to illustrate the area where the gaps are intended. Interfaces between the portions which might otherwise be vertical or close to vertical were constructed at a slightly shallower angle, such as, but not limited to, about 10 degrees, about 15 degrees or about 20 degrees away from vertical, and preferably about 15 degrees away from vertical, to avoid shadowing of the writing beam that would tend to reduce the integration during printing of the portion 94.

As illustrated in FIG. 10a, the portion 90 further includes the manipulator aperture 40. Thus, S150 also includes forming the manipulator aperture 40, when included in the design of the nerve cuff 5. Moreover, S150 may also include, after the portion 90 is fabricated, removing the same from the lithography system and submerging the portion 90 in a solvent for a period of time to rinse away unpolymerized photopolymer, and by subsequent rinsing in a mild solvent with low surface tension. For example, the period of time may be 20 or more minutes. The submerging may also wait until after the electrodes are installed on portion 90.

At S155, the electrodes are laid into the open channels 92 as illustrated in FIG. 10b.

These partially completed electrode apertures (e.g., open channels 92) allow for suitable electrodes 100 to be laid as illustrated in FIG. 10b. Where pairs of substantially aligned electrode apertures are provided as discussed above and as shown in FIGS. 10a-10d, each electrode may be sufficiently long to pass all the way across the partly completed nerve cuff, lying in corresponding aligned open channels. In an aspect of the disclosure, the electrodes are aligned such that they are parallel to each other.

In an aspect of the disclosure, the electrodes have a high-tensile strength and highly flexible electrode material. For example, carbon nanotubes can be used. The electrodes may be aligned using an alignment tool and a microscope to ensure the electrodes to extend through the cuff opening (nerve passage). The ends of the electrode may be held down during alignment.

The substrate (and uncompleted nerve cuff) is loaded back into the lithography system for the fabrication of portion 94 of the cuff. The system is optically aligned after the substrate (and uncompleted nerve cuff) is loaded and prior to fabrication of portion 94.

At S160, as illustrated in FIG. 10c, portion 94 of the nerve cuff 5 is then constructed on portion 90, thereby completing the electrode apertures with the electrodes 100 in-situ. FIG. 10c is an exploded view of the manufacturing of the nerve cuff 5. Specifically, the portions 90 and 94 are shown separately, however, in practice, portion 94 is formed directly on portion 90. For example, the printing of the portion 94 begins at the roughened surfaces 96 of the portion 90. After the portion 94 is completed, the unpolymerized photopolymer may be washed away in a solvent (as described above), leaving the completed nerve cuff structure as shown in FIG. 10d, as S165.

The nerve cuff is subsequently removed from the substrate.

In the example nerve cuff 5 of FIGS. 10a-10d, the portion 90 includes a 60 μm diameter manipulator aperture 40 for use as a fixation point for a needle or rod manipulator tool during use, for example during implantation by a surgeon, and FIG. 10d shows a completed cuff which incorporates two hinged flaps 50 similar to those illustrated in FIG. 4. The manipulator aperture 40 having a 60 μm diameter is also described as an example. The diameter may be changed as needed. While FIGS. 10a-10d show fabrication of a nerve cuff having two hinged flaps similar to those illustrated in FIG. 4, the method of construction or manufacture illustrated FIG. 10a-10d may be used to construct other nerve cuffs with electrodes having a design in accordance with aspects of the disclosure, such as, the nerve cuff illustrated in FIGS. 1, 2 and 4-9.

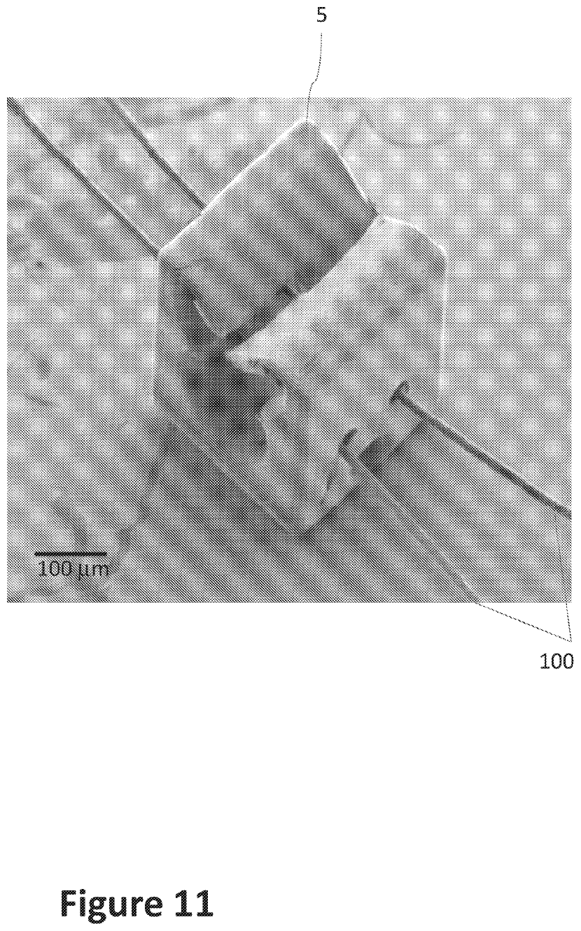

FIG. 11 illustrates an electron micrograph image of a nerve cuff 5 having electrodes 100 fabricated using the above method. This nerve cuff 5 has dimensions of about 300 μm in each direction.

The nerve cuff 5 was constructed using a two-photon direct-write 3D lithography system developed by Nanoscribe Photonic Professional GT (Nanoscribe GmbH, Eggenstein-Leopoldshafen, Germany) (an example of a stereolithography system). This lithography system was used to scan a focused laser beam (λ=780 nm) into a droplet of commercially available, UV-curable polymer (such as “IP-Dip”, “IP-S”, or “IP-L 780”, also available from Nanoscribe GmbH, Eggenstein-Leopoldshafen, Germany).

Galvanometer scanning mirrors or other optics are used to control the position of the laser focal point in a writing plane within the droplet, and a piezo actuator is used to move the stage along the optical axis to write subsequent layers. Selection of the optical power and scan speed allows for the construction of devices with features as small as 100 nm over a writing area of −300 μm in each dimension.

The nerve cuffs were printed using a 50 mm/s linear scan speed and with the optical power set to 65% of full scale (˜120 mW average power). It is noted that the scan speeds and optical powers described herein are only examples of the scan speeds and optical powers that may be used. Other scan speeds and optical powers may be used and may depend on the device or system used for the lithography and/or photopolymer material as understood by a person of ordinary skill in the art.

The solvent bath was 20 minutes (e.g., Propylene glycol methyl ether acetate, available from Sigma-Aldrich Co., St. Louis, Mo.) to rinse away unpolymerized photopolymer, and by subsequent rinsing in a mild solvent with low surface tension (for example 3M Novec 7100 Engineered Fluid, available from 3M, St. Paul, Minn.).

The electrodes were provided as carbon nanotube threads (CNTs) which provide a high-tensile strength and highly flexible electrode material. To align the electrodes into the open channels 92, they were suspended across an alignment tool, in particular a fork also fabricated using 3D printing, the alignment tool being attached to a motorized three-axis stage (such as the 3DMS, Sutter Instrument, Novato, Calif.). A dissection microscope was used to carefully align the CNT electrodes over the open channels 92 and monitor while the CNT electrodes were lowered into the channels so as to ensure they spanned the cuff opening (as illustrated in FIG. 10b). The ends of the electrodes were held down using double-sided tape placed on the periphery of the substrate on which the cuff was being fabricated.

Other techniques for fabricating the described nerve cuffs may be used such as moulding, for example using moulding, etching, and a variety of other known techniques including various micromachining techniques for fabricating small objects in a variety of materials including polymers, ceramics, metals, and semiconductors.

In order to improve biocompatibility of the described nerve cuffs 5 for implant into humans or animals, a UV-curable polymer or other material used for construction of the cuff may be chosen which has improved biocompatibility properties, and/or a biocompatible material such as parylene may be used to coat the nerve cuffs.

Electrodes suitable for combination with the nerve cuffs 5 described herein may comprise or be formed from various different materials, such as carbon nanotube fibres, and wires made of metals or semiconductors, or thin film electrodes such as polyimide with exposed gold, platinum, or other materials. The electrodes may typically have diameters which depend to some extent on the size of the nerve cuff and the intended application, and also upon the required tensile strength, but may typically be less than about 1000 μm, less than about 200 μm, and optionally less than about 50 μm or even less than about 10 μm.

For example, nerve cuffs, in accordance with aspects of the disclosure, have been constructed having carbon nanotube fibers therein, the fibers having diameters in the range of about 10-50 μm. Suitable such fibres are discussed in, for example in Chengmin Jiang et al., “Macroscopic Nanotube Fibers Spun from Single-Walled Carbon Nanotube Polyelectrolytes”, ACS Nano, Vol. 8, 9107-9112, 2014 and Flavia Vitale et al., “Neural Stimulation and Recording with Bidirectional, Soft Carbon Nanotube Fiber Microelectrodes”, ACS Nano, 2015, 9 (4), pp 4465-4474, which are incorporated herein by reference. Electrodes combined into a nerve cuff 5 during fabrication or subsequently may be at least several millimeters, and sometimes several centimeters long, so as to facilitate subsequent electrical connection.

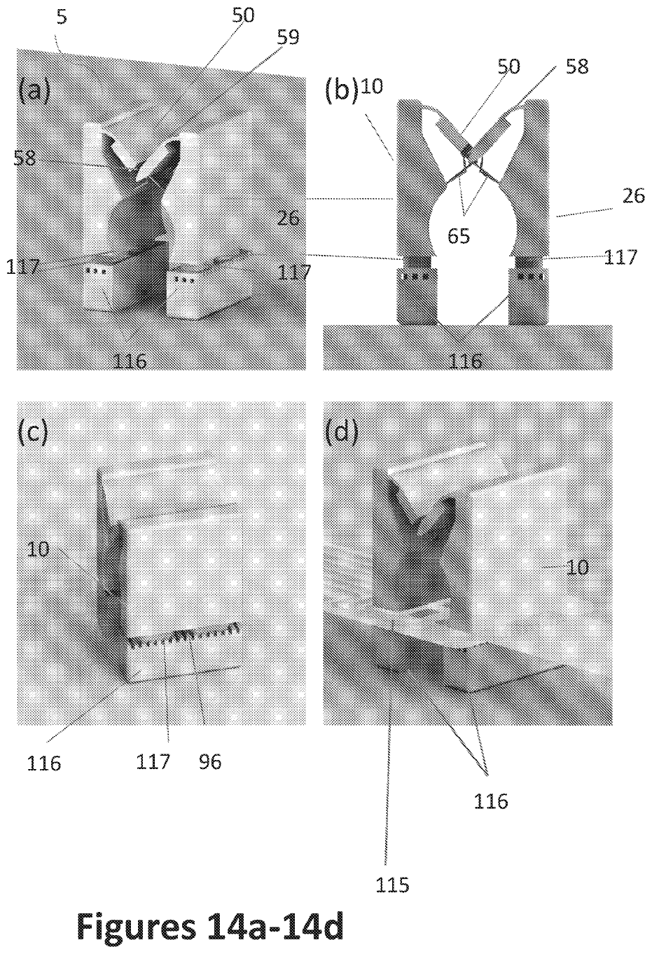

Nerve cuffs, in accordance with aspects of the disclosure, have also been constructed having thin film electrodes therein, the thin film electrodes have thicknesses in the range of about 8-15 μm. FIGS. 14a-14d illustrates different views of a nerve cuff configured for using thin film electrodes 115.

Suitable such thin film arrays are discussed in, for example Kee-Keun Lee et al, “Polyimide-based intracortical neural implant with improved structural stiffness,” Journal of Micromechanics and Microengineering, 2003, 14 (1) and S. Cogan, “Biomedical Device with a Protective Overlayer,” 1998, U.S. Pat. No. 5,755,759, which are also incorporated by reference.

For descriptive purposes, the nerve cuff 5 illustrated in FIG. 14a-14d has hinged flaps 50 confine the nerve 44 within the nerve passage 16 with interlocking teeth 56 (similar to those depicted FIG. 4). While FIGS. 14a-14d have hinged flaps as the gating structures, any of the gating structures described above can be incorporated into the nerve cuff 5 depicted in FIGS. 14a-14d. FIG. 14a is a perspective view of the nerve cuff (without the thin film electrode). FIG. 14b is a plan view of the nerve cuff (without the thin film electrode). FIG. 14c is a side view showing the roughened surfaces on the segments in accordance with aspects of the disclosure. FIG. 14d is a perspective view of the nerve cuff having the thin film electrode installed.

The nerve cuff 5 can have a single base 24 (similar to the base depicted in FIG. 1, but in the example shown in FIGS. 14a-14d, the base is split into two base segments 116 (first and second segments). As illustrated in FIGS. 14a-14d, the first and second segments are connected to the cuff body 10 with pillars 117. As illustrated, the nerve cuff 5 has four pillars 117, two on each side 26. However, the number of pillars on each side may depend on the number of vias or openings in the thin film electrode.

Since the base is split into two separated segments, the sides 26 are not directly connected to each other. Rather, the sides 26 are separate elements held semi-rigidly in place by an electrode assembly 115. Specifically, the rigidity of the electrode 115 allows the cuff body 10 to remain rigid.

As depicted in FIG. 14d, the thin film electrode 115 is located between the cuff body 10 and the base segments 116.

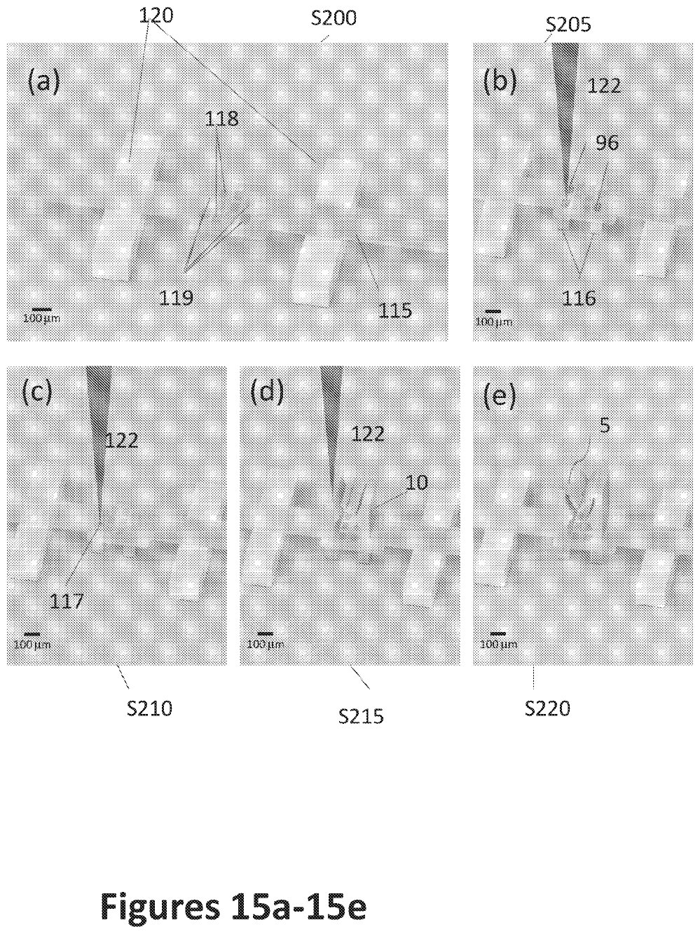

FIGS. 15a-15e illustrate a method of constructing or manufacturing the nerve cuff illustrated in FIGS. 14a-14d. In accordance with aspects of the disclosure, the method may use a stereolithography process. The method may use a similar lithography machine as described above. For example, a Nanoscribe Photonic Professional GT system may be used. A photopolymer such as photoresist IP-dip also available from Nanoscribe may also be used.

As illustrated in FIG. 15a, the thin film electrode 115 comprises one or more contacts. The contacts are designed as conductive traces made of gold (or platinum or other suitable material) within a film of polyimide, thus the traces are insulated by the polyimide, and the conductive layer is exposed as at pad sites 118 only within the nerve passage to deliver or draw current from the nerve.

The electrode 115 also has four vias 119 (which correspond to the number of pillars 117 in the nerve cuff 5 illustrated in FIG. 14a-14d). The number, shape, and size of the vias may change, which in turn may drive a change in the number, shape and size of the corresponding pillars 117. Additionally, the exact size, geometry and number of channels of the electrode can be varied. As illustrated in FIG. 15a, the contacts are adjacent to the vias and are located in an area where, when installed in the nerve cuff 5, the contacts (gold pads) will be within the nerve passage 16, such that the contacts can be proximal to the peripheral nerve.

As described above, the purpose of the base segments 116 of the nerve cuff 5 is to seal the electrode 115 between themselves and the cuff body 10. The purpose of the pillars 117 is to connect the base segments 116 and the cuff body 10, and to prevent the electrode 115 from sliding out of the nerve cuff 5.

According to aspects of the disclosure, the nerve cuff 5 (illustrated in FIGS. 14a-14d) may be constructed in multiple steps.

Referring to FIG. 15a, at S200, the electrode 115 is first placed over a silicon base or other solid substrate at a fixed distance using thin tape 120 or another thin adhesive. The fixed distance defines a dimension of the segment. As oriented in FIGS. 14a-14d, the height of the segments 116 is selected such that the segments 116 can provide the necessary force to seal the electrode 115 between themselves and the cuff body 10.

Prior to being inserted into the machine, the substrate and electrode 115 is submerged in a photopolymer such as the photoresist IP-dip and the entire assembly is inserted into a stereolithography system such as Nanoscribe Photonic Professional GT system.

At S205, the base segments 116 are printed under the thin film electrode 115, starting from the surface of the substrate and extending towards the thin film electrode 115 as illustrated in FIG. 15b. A focused laser beam 122 (represented in FIGS. 15b-15d as an elongated triangle) is used to cure the photopolymer. FIG. 15b shows the laser beam 122 at a specific voxel along the scan path for forming the segments. As shown in FIG. 15b, the focused laser beam 122 is directed downwards towards the photopolymer. It is noted that the thin film electrode 115 is sufficiently thin such that it is largely transparent to the laser beam, enabling the energy of the beam to reach the photopolymer material without having to change the orientation of the laser source. The focused laser beam is scanned throughout the area in which the photopolymer material is to form the segments 116, layer-by layer. In an aspect of the disclosure, the base segments comprise roughened surfaces 96 at the top of the base segments 116. The roughened surface 96 increases adhesion between the base segments 116 and the oval pillars 117. The roughened surface 96 is created by controlling the laser source to selectly emit the laser beam in areas for curing and not emitting the laser beam in areas where gaps or spaces are desired.

As described above, linear scan speed and optical power of the device can be controlled to print the cuff. For example, in accordance with aspects of the disclosure, the base segments 116 may be printing using a linear scan speed of about 110 mm/s, about 115 mm/s, about 120 mm/s, about 125 mm/s or 130 mm/s. Additionally, for example, in accordance with aspects of the disclosure, the optical power may be set to about 95%, about 100%, about 105%, about 110% or about 115% of full scale for the laser beam. The cuff 5 depicted in FIG. 15b were printed using about 120 mm/s linear scan speed and with the optical power set to about 105% of full scale for the laser beam 122.

At S210, the pillars 117 are formed. In an aspect of the disclosure, to form the pillars 117, the power may be reduced to about 70%, about 75%, about 80%, about 85% or about 90%. For example, in accordance with aspects of the disclosure, the pillars 117 depicted in FIG. 15c were printed using an optical power set to about 82% of full scale. In the nerve cuff illustrated in FIG. 14a-14d, the pillars are oval, but the pillars can be other shapes. The oval pillars are printed through the vias 119 in the thin film electrode, starting from the roughened surface 96. FIG. 15c shows the laser beam 122 at a specific voxel along the scan path for forming the pillars.

At S215, additional portions of the cuff are then printed on the pillars 117 and thin film electrode 115. The additional portions are formed in a similar manner using the laser beam 122 to cure the photopolymer layer-by-layer. FIG. 15c shows the laser beam 122 at a specific voxel along the scan path for forming the cuff body 10. The sides 26 are printed along with the flaps 50. While FIG. 15d does not show a completed cuff, the top and flaps are completed in S215.

Similar to above, the nerve cuff 5 on the silicon substrate with the electrode 115 integrated is then submerged into a strong solvent (e.g., propylene glycol methyl ether acetate) for a period of time, e.g., 20 minutes, to remove the unpolymerized photoresist and then into a mild solvent with low surface tension (Novec 7100) to remove the excess strong solvent and any unpolymerized photoresist residues at S220 (the completed nerve cuff 5 is shown in FIG. 15e).

In accordance with aspects of the disclosure, the electrode may be electrically insulated except close to a specific or a target area. This allows for the application of an electrical signal to a specific or target area of the peripheral nerve, or collect an electrical signal from the specific or target area of the peripheral nerve retained with the nerve passage 18 of a described nerve cuff. To this end, the electrodes may be insulated using a material such as a polymer or other coating, such as parylene or polyimide, with the coating either then being removed from, or never applied to the electrode close to the target area.

The fabrication method as depicted in FIGS. 15a-e allows for the incorporation of insulated electrodes within the nerve cuff 5.

Another example of fabricating a nerve cuff 5 with suitably insulated electrodes proceeds by coating a nerve cuff already combined with electrodes with an insulating coating, and then ablating the insulating coating from at least part of each electrode where exposed within the nerve passage. According to aspects of the disclosure, the method may comprise:

(a) First the portion 90 of the nerve cuff is printed on a silicon or other base;

(b) Uncoated electrodes, such as carbon nanotube fibers, are located into the channels 92 as shown in FIG. 10b;

(c) The portion 94 of the cuff is printed;

(d) The nerve cuff is removed from the silicon base and suspended in air by the electrodes, with the electrodes being kept separate from each other;

(e) The nerve cuff and the electrodes are both coated with a layer of parylene, for example to a thickness of about 6 μm;

(f) The nerve cuff is then placed on a small tungsten needle inserted into the manipulation aperture;

(g) The nerve cuff is turned sideways so that the electrodes are visible through the end of the nerve passage 18;

(h) A suitable laser (such as, for example, a ˜1 Watt, tunable femtosecond laser) is focussed onto the near side electrode within the nerve passage 18;

(i) The laser repeatedly follows a suitable path to remove the coating, for example with a path repetition about 300 times at about 75% power and with a dwell time at each location in the path of about 4 milliseconds;

(j) The nerve cuff is turned around to access the other electrode from the other end of the nerve passage and steps (h) and (i) are repeated for the second electrode.

In other aspects of the disclosure, the electrodes may be similarly coated, for example using parylene, and then a small length or region of each electrode may be processed for example by laser ablation to remove the coating from a region which is then aligned into the nerve passage of a nerve cuff either during or after fabrication of the cuff. In an aspect of the disclosure, the coating/ablation may be performed during S155 of the fabrication process while the electrode is exposed (prior to forming portion 94).

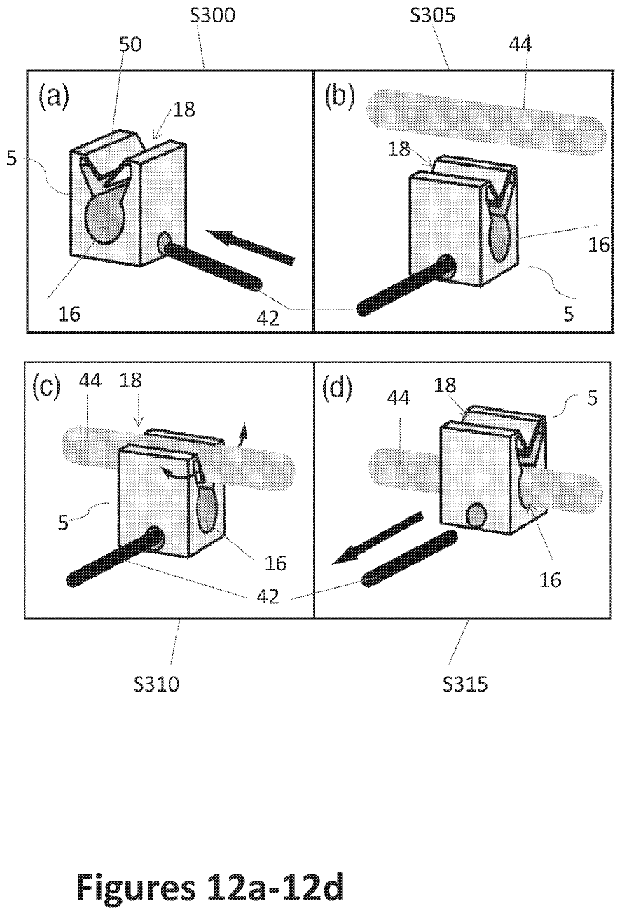

Referring now to FIGS. 12a-12d, a method in accordance with aspects of the disclosure of applying a described nerve cuff to an in vivo peripheral nerve is shown. While the nerve cuff 5 illustrated in FIGS. 12a-12d comprises a pair of hinged flaps 50 to act as gating structures, similar to those of FIG. 2, the nerve cuff used in this or similar methods could have a variety of gating structures or other features to assist in moving the nerve into and/or retaining the nerve in the nerve channel 16 of the nerve cuff 5. For simplicity and clarity, electrodes, electrode apertures and similar have not been illustrated in FIGS. 12a-12d, although of course a variety of such elements can be included as described elsewhere in this document.

In FIG. 12a, at S300, a manipulator tool 42 such as needle is inserted into manipulator aperture 40 for handling the nerve cuff 5. The direction of insertion is represented in FIG. 12a as a direction arrow. In FIG. 12b, at S305, the nerve cuff 5 is brought into close proximity to a peripheral nerve 44. For example, the nerve cuff 5 may be moved using the manipulator tool 42, and the nerve axis or the nerve passage 16 of the cuff and the peripheral nerve 44 are brought into line so as to be approximately parallel.

In FIG. 12c, S310 the peripheral nerve 44 and the nerve cuff 5 are brought together so that the nerve starts to enter into the entry channel 18 of the nerve cuff 5, e.g., still using the manipulator tool 42. The peripheral nerve 44 is then obliged through the entry channel 18, by movement of the cuff, movement of the nerve, or both. During this stage, the nerve is urged past any gating structures provided in the entry channel 18. For example, in the case of a nerve cuff in which resilient hinged flaps 50 are provided as illustrated in FIGS. 2 to 4, these hinged flaps 50 are pressed downwards and towards the sides 26 of the cuff body 10 to allow the nerve to pass into the nerve passage 18. This is shown in FIG. 12c by curved arrows representing the direction of movement of the hinged flaps 50.

If one or more baffle structures such as those illustrated in FIGS. 5 and 6 are provided then the nerve 44 presses past these baffle structures, through deformation of the nerve 44 and/or deformation of the baffle structures, again to pass into the nerve passage 16.

In FIG. 12d, the peripheral nerve 44 is located within the nerve passage 16. Since the peripheral nerve 44 is in the nerve passage 16 and no longer in the entry channel 18, any moving hinged flaps 50 or similar structures return under their own resilience to a neutral position, to thereby resist movement of the peripheral nerve out of the nerve passage 16.

Similarly, appropriate sizing of the nerve passage 16 to match the undeformed nerve cross section, a suitable neck 20 between the nerve passage 16 and the entry channel 18, and any baffles, e.g., teeth 70 or ridges 72, in the entry channel, may help to retain the nerve 44 in the nerve passage 16.

If any lid structures 80 such as those shown in FIGS. 7 and 8 are provided, then these may now be closed also to resist escape of the nerve 44 from the nerve cuff 5. Finally, at S315, the manipulation tool 42 is removed from the nerve cuff 5. For example, in this case, by withdrawing a needle from the manipulation aperture 40. The removal direction is represented by a directional arrow in FIG. 12d.

Installation of the nerve cuff 5 on the nerve 44 is now complete, although further steps may then be needed to complete any electrical connections to the nerve cuff 5 if not already made. If the cuff remains in situ for some time, tissue growth in and around the cuff will typically take place to help further secure retention of the nerve 44 within the nerve cuff 5.

The described nerve cuffs are intended primarily for use on smaller peripheral nerves, for example on such nerves having a diameter of about 1 mm or less, including much smaller nerves having a diameter down to about 100 μm or less, although the cuff may be constructed for use with larger nerves having diameters of greater even than about 1 mm if desired.

Reference herein to a nerve or a peripheral nerve, it is to be understood that this may also refer to a branch of a peripheral nerve, and may also refer to a part of a nerve (including part of a branch) which has been separated out, for example a dissected fascicle or other subcomponent of a nerve, in a manner which permits only that part of the nerve to be introduced into the nerve cuff. Additionally, the nerve cuff 5 may be applied to ganglia, containing the cell bodes of the nerve.

The nerve cuff 5 may be characterized by a diameter of the nerve passage 16 which may therefore be about 10 mm or less, about 5 mm or less, about 2 mm or less, about 1 mm or less, about 500 μm or less, about 200 μm or less, or about 100 μm or less. This diameter may be taken, for example, as the diameter of the nerve passage 16 in a direction transverse to the direction in which the nerve is introduced through the entry channel 18 that is in the direction of alignment of the electrodes in the accompanying figures.

The nerve cuff 5 may also or instead be characterized by a largest dimension of the nerve cuff in a direction transverse to the nerve passage 16, which could for example be about 10 mm or less, about 5 mm or less, about 2 mm or less, about 1 mm or less, about 500 μm or less, about 200 μm or less, or about 100 μm or less.

Some particular uses of the nerve cuffs described herein include attachment to the carotid sinus nerve (CSN) in humans to apply block and treat type 2 diabetes. The CSN is about 1 mm in diameter in humans so is difficult to provide an electrical connection to using prior art nerve cuffs. Larger nerves on which the nerve cuffs described herein may be used include the vagus nerve in humans (about 5 mm in diameter) that can be used to treat epilepsy, depression, and rheumatoid arthritis, including the pulmonary branch of the vagus nerve to treat asthma.

Nerve cuffs described herein can also be used for neuroprosthesis to restore movement by electrically stimulating nerves involved in motor control, for example by cuffing the sciatic nerve which has a diameter of about 1 cm in humans. Disease applications include, for example, correcting foot drop, for restoring walking after spinal cord injury, and for use in the arms of a patient to restore grasping. The described nerve cuffs can also or instead be used to provide sensory feedback for control of robotic prostheses, such as artificial arms, hands or lower extremities, and for other uses in which electrical signals of the nerve are detected. Another use is for blocking peripheral nerves for treatment of phantom limb and pain.

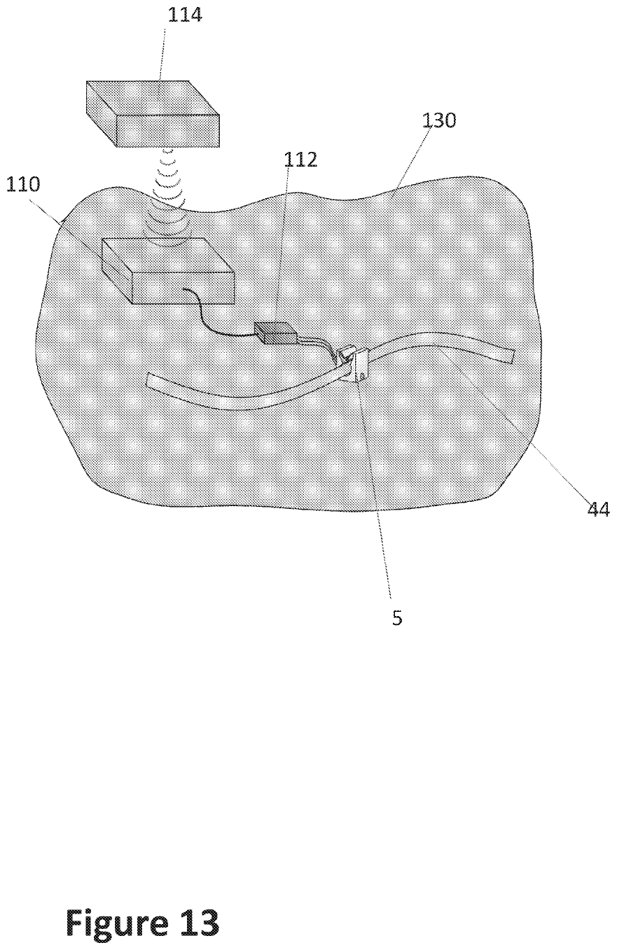

FIG. 13 illustrates a system 1300 comprising a nerve cuff 5 in accordance with aspects of the disclosure installed in a human or animal subject. FIG. 13 illustrates the nerve cuff 5 applied to a peripheral nerve 44, for example using the method illustrated in FIGS. 12a-12d. In this case the nerve cuff 5 has been applied in-vivo in a human or animal subject, and an incision in the tissue 130 of the subject used to apply the cuff has been closed (not shown in FIG. 13). However, it may be desirable in some cases to use a nerve cuff 5 on a peripheral nerve 44 without closing the incision, for example where a treatment, a diagnosis or an experiment is to last only a short time.

The system 1300 further comprises a driving unit 110 and a control unit 114. In the arrangement of FIG. 13, a driving unit 110 has also been implanted in the subject. The driving unit 110 is electrically connected to the electrodes of the nerve cuff 5. In an aspect of the disclosure, the driving unit 110 is electrically connected to an electrode using a connector element 112. In some cases of course, it may be desirable for the driving unit 110 to be located outside of the subject for example using one or more electrical connectors passing through the skin. Even with a driving unit 110 located within the subject, it will frequently be necessary to use the control unit 114 external to the patient to transmit and/or receive data and/or power to the driving unit 110. In an aspect of the disclosure, the control unit 114 may power the driving unit 110 inductively, via an inductive coupling.

The driving unit 110 may fulfill a variety of functions depending on the intended use of the nerve cuff 5, for example by supplying one or more stimulation signals to the electrodes so as to stimulate the nerve 44 in some way, including providing a stimulation signal to block the nerve 44, and/or reading one or more electrical signals from the nerve 44.

For example, two nerve cuffs, each containing two conducting electrodes, were placed on a peripheral nerve of an animal subject, separated by a distance of about 10 mm. The upper cuff was connected to a “PlexStim” stimulator (supplied by Plexon, Dallas, Tex.) for stimulation (driving unit). The lower cuff was connected to a “Medusa” Preamplifier (supplied by Tucker Davis Technologies, Alchua, Fla.) for recording. The recorded signal was band-pass filtered between 1 Hz and 20 kHz and sampled at 24.414 ksamples/sec. The nerve was subjected to a series of increasing amplitude biphasic current-controlled pulses until a response was seen on the recording electrodes. The stimulation threshold was recorded. Then the amplitude was increased and the nerve response activity was recorded. This procedure was repeated to study the effect on pulse width, as well.

After stimulation-triggered-responses were gathered, a small amount of bupivacaine, a sodium channel blocker, was dripped onto the nerve at the stimulation site to prevent activation. This technique ensured that the evoked responses were neural and not due to signal contamination (e.g. EMG artefact from neighbouring muscles). The above procedure was then repeated to verify that the nerve activity was impeded at a range of currents and pulse widths.

Another example use of the described nerve cuffs 5 is for velocity sensitive recording in which a series of electrodes spaced along a nerve 44 held within the nerve passage 16 provide a series of longitudinally spaced recordings of electrical activity in the nerve 44. A goal may be to identify axon populations by velocity and therefore, fiber diameter. Certain signal modalities have different diameter nerves. For instance, pain and temperature information is transmitted on small diameter fibers whereas sensation of limb location is through large diameter fibers. The vagus nerve consists of different fiber types which are assumed to control different functions as well (e.g. large fibers are pulmonary stretch afferents and small diameter might be gastrointestinal functions). For this sort of application, a nerve cuff 5 which is relatively elongate in the direction of the nerve may be desirable, for example to achieve a total spacing between first and last electrodes in a series of several mm.

Although the nerve cuff has been described as being used for a nerve, the nerve cuff may also be used with other internal body tissue such as, e.g., smooth muscles, striated muscles, arteries, veins, ligamental tissues, connective tissues, cartilage tissues, bones, or other similar body tissues, structures or organs.

Although particular aspects have been described, it will be apparent to the skilled person that a variety of modifications and alternatives may be implemented without departing from the spirit and scope of the disclosure.

What is claimed is:

1. A nerve cuff configured to retain one or more electrodes proximal to a peripheral nerve when retained within the nerve cuff, the nerve cuff comprising:
a rigid cuff body having opposite first and second ends;
a nerve passage extending between said first and second ends of the rigid cuff body, the nerve passage configured to retain the peripheral nerve; and
an entry channel extending between said first and second ends of the rigid cuff body, the entry channel guiding the peripheral nerve towards the nerve passage, the nerve cuff being configured to inhibit the peripheral nerve, when retained in the nerve passage, from being removed from the nerve cuff.

2. The nerve cuff of claim 1, wherein the nerve cuff is further configured to bias the peripheral nerve towards the nerve passage.

3. The nerve cuff of claim 1 or claim 2, wherein the rigid cuff body comprises opposite first and second sides extending between said first and second ends, a portion of the first side and a portion of the second side form sidewalls of the entry channel, wherein the sidewalls of the entry channel are angled such that an opening to the nerve passage from the entry channel is narrower in a transverse direction to the nerve passage than an opening to the entry channel to an exterior in the transverse direction.

4. The nerve cuff of claim 3, wherein another portion of the first side and another portion of the second side form sidewalls of the nerve passage, the sidewalls of the nerve passage being curved, wherein the opening to the nerve passage from the entry channel is narrower in a transverse direction to the nerve passage than a maximum distance between sidewalls in the transverse direction.

5. The nerve cuff of claim 1, wherein the nerve cuff is an integrally formed unit substantially of a single material.

6. The nerve cuff of claim 1, wherein the nerve cuff is coated with another material.

7. The nerve cuff of claim 5, wherein the single material is a photopolymer.

8. The nerve cuff of any preceding claim, wherein a largest dimension of the cuff in the transverse direction to the nerve passage is less than 10 mm.

9. The nerve cuff of any of claims 1 to 7, wherein a diameter of the nerve passage is less than 10 mm.

10. The nerve cuff of any preceding claim, wherein the rigid cuff body further comprises one or more electrode apertures extending through said rigid cuff body to the nerve passage for accepting said one or more electrodes.

11. The nerve cuff of claim 10, wherein the rigid cuff body comprises opposite first and second sides extending between said first end and said second end, and the one or more electrode apertures comprise at least one pair of electrode apertures, where each pair of the at least one pair of electrode apertures are arranged such that a respective electrode aperture of the pair pass through a different one of said first side and said second side, and each pair is axially aligned so that a single electrode can be retained in the electrode apertures of a pair.

12. The nerve cuff of claim 11, wherein the at least one pair of electrode apertures is a plurality of said pairs of electrode apertures, the plurality of said pairs of electrode apertures are distributed along the rigid body between said first and second ends so that multiple electrodes can be provided proximal to distributed locations along a peripheral nerve when retained within the nerve passage.

13. The nerve cuff of any preceding claim, further comprising one or more said electrodes arranged proximal to a peripheral nerve when retained in the nerve passage.

14. The nerve cuff of claim 13, wherein at least one of the one or more electrodes comprises a metallic wire.

15. The nerve cuff of claim 13, wherein at least one of the one or more electrodes comprises carbon nanotubes.

16. The nerve cuff of any of claims 13 to 15, wherein at least one of the one or more electrodes has a diameter in a range from 1 to 1000 μm.

17. The nerve cuff of any of claims 13 to 16, wherein at least one of the or more electrodes is at least partly covered in an insulating layer where the at least one electrode is external to the nerve cuff, and the insulating layer is absent from at least a portion of the at least one electrode within the nerve cuff.

18. The nerve cuff of claim 2, wherein the entry channel is at least partly defined by entry channel side walls which approach the nerve passage such that the entry channel and the nerve passage define a neck between them, the neck being narrower than the nerve passage.

19. The nerve cuff of any preceding claim further comprising one or more gating structures configured for one or more of restrain movement of a peripheral nerve through the entry channel in a direction away from the nerve passage when the peripheral nerve is retained and to bias movement of a peripheral nerve through the entry channel in a direction towards the nerve passage.

20. The nerve cuff of claim 19, wherein the one or more gating structures comprise one or more baffles protruding into the entry channel.

21. The nerve cuff of claim 20, wherein the one or more baffles are inclined towards the nerve passage.

22. The nerve cuff of any of claims 19 to 21, wherein the one or more gating structures comprise a pair of opposing hinged flaps inclined towards the nerve passage and having proximal tips, the pair of flaps being arranged to separate when urged in a forward direction to permit a peripheral nerve to pass between the tips when moving through the entry channel towards the nerve passage.

23. The nerve cuff of claim 22, wherein the pair of flaps is arranged such that when urged in a reverse direction the tips engage to limit a reverse movement.

24. The nerve cuff of claim 23, wherein the tips interlock when engaged.

25. The nerve cuff of any of claims 19 to 24, wherein the one or more gating structures comprise a lid, arranged such that the lid can be moved between an open configuration to allow a peripheral nerve to pass into the entry channel towards the nerve passage, and a closed configuration in which the entry channel is blocked by the lid to prevent exit of a peripheral nerve out of the entry channel away from the nerve passage.

26. The nerve cuff of claim 25, wherein the lid is hinged to provide movement between the open and closed configurations, and the nerve cuff further comprises a catch to secure the lid in the closed configuration.

27. The nerve cuff of any preceding claim further comprising a manipulator aperture in the rigid cuff body, the manipulator aperture being arranged to accept a manipulator tool for handling the nerve cuff.

28. The nerve cuff of claim 27, wherein the manipulator aperture extends in a direction transverse to the nerve passage.

29. The nerve cuff of claim 27 or claim 28, wherein manipulator aperture extends all the way across the rigid cuff body such that a manipulator tool can be accepted from either end of the manipulator aperture.

30. The nerve cuff of any of claims 27 to 29, wherein the manipulator aperture intersects with the nerve passage.

31. The nerve cuff of any preceding claim further comprising a manipulator pad arranged for securing to a manipulator tool, the manipulator pad being coupled to the rigid cuff body by a frangible connection.

32. The nerve cuff of any of claims 1 to 9, further comprising a first segment and a second segment, the first segment and the second segment being connected to the rigid cuff body via connection pillars.

33. The nerve cuff of claim 32, further comprising a thin film electrode, the thin film electrode having a plurality of vias, each of the plurality of vias dimensioned such that a corresponding connection pillar of the connection pillars extends through the via, wherein the thin film electrode is disposed between the rigid cuff body and the first segment and the rigid cuff body and the second segment.

34. The nerve cuff of claim 32 or claim 33, wherein the thin film electrode comprising one or more electric contacts disposed in the nerve passage and configured to be proximal to the peripheral nerve when the peripheral nerve is in the nerve passage.

35. The nerve cuff of any of claims 32 to 34, further comprising one or more gating structures configured for one or more of restrain movement of a peripheral nerve through the entry channel in a direction away from the nerve passage when the peripheral nerve is retained and to bias movement of a peripheral nerve through the entry channel in a direction towards the nerve passage.

36. The nerve cuff of any of claims 32 to 35, wherein the one or more gating structures is selected from a group consisting of one or more baffles protruding into the entry channel, a pair of opposing hinged flaps inclined towards the nerve passage and having proximal tips and a lid.

37. The nerve cuff of claim 36, wherein the one or more gating structures is the one or more baffles, the one or more baffles being inclined towards the nerve passage.

38. The nerve cuff of claim 36, wherein the one or more gating structures is the pair of opposing hinged flaps inclined towards the nerve passage and having proximal tips, the pair of flaps being arranged to separate when urged in a forward direction to permit a peripheral nerve to pass between the tips when moving through the entry channel towards the nerve passage and arranges such that when urged in a reverse direction the tips engage to limit a reverse movement.

39. The nerve cuff of claim 36, wherein the one or more gating structures is the lid, the lid being arranged such that the lid can be moved between an open configuration to allow a peripheral nerve to pass into the entry channel towards the nerve passage, and a closed configuration in which the entry channel is blocked by the lid to prevent exit of a peripheral nerve out of the entry channel away from the nerve passage.

40. The nerve cuff of claim 39, wherein the lid is hinged to provide movement between the open and closed configurations, and the nerve cuff further comprises a catch to secure the lid in the closed configuration.

41. A method of constructing the nerve cuff as set out in any of claims 1 to 31, the nerve cuff being configured to retain one or more electrodes proximal to a peripheral nerve when retained within the nerve cuff, the method comprising forming said nerve cuff as an integral unit, the nerve cuff comprising at least the rigid cuff body having the opposite first and second ends, the nerve passage extending between said first and second ends, the nerve passage configured to retain the peripheral nerve, and the entry channel extending between said first and second ends, the nerve cuff being configured to inhibit the peripheral nerve, when retained in the nerve passage, from being removed from the nerve cuff.

42. The method of claim 41 comprising forming some or all of said nerve cuff using a 3D printing technique.

43. The method of claim 42, wherein said 3D printing technique is a stereolithography or direct laser writing technique in which a light field or scanned laser spot is used to write the cuff structure into a photopolymer.

44. The method of any of claims 41 to 43, wherein the rigid cuff body further comprises one or more electrode apertures extending through said rigid cuff body to the nerve passage for accepting said one or more electrodes, and the method comprises:
forming a portion of the nerve cuff, the portion comprising one or more open channels each corresponding to an uncompleted one of said electrode apertures;
laying one or more electrodes into said open channels; and
forming a further portion of the nerve cuff on the portion thereby closing the one or more open channels to complete said electrode apertures containing said one or more electrodes.

45. The method of any of claims 41 to 44, wherein the electrodes comprise carbon nanotube threads.

46. The method of claim 44, wherein the rigid cuff body further comprises a manipulator aperture, wherein the forming of the portion of the nerve cuff, further comprising forming the manipulator aperture as a opening in the portion.

47. The method of claim 44, wherein forming the portion of the nerve cuff further comprising forming a roughened surface on the portion, the roughened surface being an interface for forming the further portion of the nerve cuff.

48. The method of claim 44, wherein the one or more electrode apertures comprises a pair of apertures, wherein the forming the portion of the nerve cuff, further comprising aligning in a direction transverse to the nerve passage, a pair of open channels, each corresponding to one of the apertures and the laying one or more electrodes into said open channels further comprising aligning in the direction transverse to the nerve passage an electrode in the pair of open channels.

49. A method of constructing a nerve cuff comprising a thin film electrode, the thin film electrode having a plurality of vias, the method comprising:
placing the thin film electrode at a set distance from a substrate, the thin film electrode being parallel to the substrate;
forming a first segment and a second segment between the thin film electrode and the substrate, the first segment and the second segment having at least a portion separate from each other in a direction transverse to the nerve passage;
forming connecting pillars through the plurality of vias on the first segment and the second segment; and
forming a rigid cuff body having opposite first and second ends and sidewalls extending between the first end and the second end, at least a portion of the sidewalls are configured and dimensioned to provide a nerve passage, the rigid cuff body also having a entry channel configured to guide the nerve to the nerve passage.

50. A method of retaining one or more electrodes at a peripheral nerve, comprising:
providing a nerve cuff as set out in any of claims 1 to 40, having at least a rigid cuff body, a nerve passage, and an entry channel, and one or more electrodes; and
moving the peripheral nerve through the entry channel and into the nerve passage so as to be retained adjacent to the one or more electrodes.

51. The method of claim 50, wherein the method is carried out using a manipulator coupled to the rigid cuff body, and further comprises removing the manipulator when the peripheral nerve is retained proximal to the one or more electrodes.

52. The method of claim 50 or claim 51, further comprising at least one of reading an electrical signal from the peripheral nerve, or passing an electrical signal to the peripheral nerve, using the one or more electrodes.
